(19) United States
(12) Patent Application Publication
(10) Pub. No.: US 2015/0258190 A1
(43) Pub. Date: Sep. 17, 2015

(54) NICOTINAMIDE AS ADJUVANT

(71) Applicant: NOVARTIS AG, Basel (CH)

(72) Inventor: Guido Grandi, Siena (IT)

(21) Appl. No.: 14/438,112

(22) PCT Filed: Oct. 25, 2013
(86) PCT No.: PCT/EP2013/072341

§ 371 Date: Apr. 23, 2015

Related U.S. Application Data
(60) Provisional application No. 61/718,373, filed on Oct. 25, 2012.

(51) Int. Cl. A61K 39/39; A61K 39/085

(52) U.S. Cl. CPC A61K 39/39; A61K 39/085; A61K 2039/55511

(57) ABSTRACT

The invention relates to methods for enhancing the protective immunity elicited by an immunogen that comprise administering an immunomnodulatory compound to a patient separately but simultaneously with, or prior or subsequent to, the administration of a vaccine. In particular, the present invention relates to the use of nicotinamide and derivatives thereof and benzamide and derivatives thereof in enhancing the protective immunity elicited by an immunogen.

This application claims the benefit of U.S. provisional application 61/718,373 filed Oct. 25, 2012, the complete contents of all of which are hereby incorporated herein by reference for all purposes.

### TECHNICAL FIELD

The present invention relates to the use of an immunomodulatory compound in enhancing the protective immunity elicited by an immunogen.

### BACKGROUND ART

Mammals have developed a very sophisticated and effective system to protect themselves against attacks from pathogens. In essence, the system involves two temporally separated cascades of events known as “innate immune responses” and “adaptive immune responses”.

Innate immunity's main role is to respond immediately (within minutes to hours) to pathogen invasion. It operates through the action of antimicrobial components (antimicrobial peptides, complement components, proteases etc.) and specialized phagocytic cells that engulf and kill invading pathogens. Pathogen recognition by the innate immune system is not pathogen-specific but rather occurs through receptors that bind structural motifs shared by different families of pathogen and are known as “Pathogen-Associated Molecular Patterns” (PAMPs). By contrast, adaptive immunity fully develops within days after pathogen invasion.

The adaptive immune response is highly pathogen-specific and typically recognises only antigens which are unique to pathogens of the same or a closely related species. The adaptive immune response is generally long-lasting, i.e. once it has been fully mounted it is capable of protecting against subsequent invasions of the same pathogen.

Both innate and adaptive immunity can be artificially stimulated to help the body in fighting an infection. Classical vaccination works by inducing an adaptive immune response against a pathogen by administering pathogen-specific antigens. Once a vaccinated individual's body encounters the pathogen, it will mount an adaptive immune response without delay.

The use of immunomodulatory compounds as adjuvants to enhance the adaptive immune response elicited by a vaccine has been described previously. For example, complete Freund's adjuvant (which contains inactivated and dried mycobateria) has been used for many decades to elicit a more potent adaptive immune response to otherwise only weakly or less immunogenic antigens.

An innate immune response can be stimulated by systemically or locally administering molecules that resemble PAMPs. This can enhance the body's capacity to neutralize a pathogen. In the last quarter century, the specific structural motifs or PAMPs on bacteria and other pathogens, which trigger an innate immune response, and the receptors to which they bind (generically referred to as pattern recognition receptors) have been identified. Toll-like receptors (TLRs) form an important subgroup of these pattern recognition receptors. TLRs induce a potent immune response by activating the expression of a wide range of proinflammatory genes including IL-1β and IL-12.

Over the past decade, many synthetically produced TLR agonists have been tested as potential adjuvants for various vaccines. So far, no TLR agonist has been approved for separate administration to a (human) patient in order to enhance the protective immunity elicited by a vaccine.

Paradoxically, immunomodulatory compounds that inhibit a key regulator of inflammation, TNF-α, and interfere with IL-1β and IL-12 production by monocytes have been found more recently to also enhance the immune response elicited by vaccines. For example, WO2007/028047 [1] discloses the use of structural and functional analogues of thalidomide such as lenalidomide and pomalidomide for reducing or inhibiting the immunosuppressive activity of regulatory T cells with the purpose of eliciting an enhanced immune response to an immunogen. Dredge et al. [2] demonstrated that the presence of pomalidomide during the priming phase strongly enhanced antitumour immunity elicited by a whole tumour cell vaccine and correlated with protection from a subsequent live-tumour challenge. However, both lenalidomide and pomalidomide have a toxicity profile that makes them unsuitable for general application. In particular, lenalidomide and pomalidomide cannot be prescribed to women who are pregnant or able to conceive. The use of these compounds in the paediatric patient population is also limited due to their various side effects.

US2009/0074815 [3] describes the use of γ-D-glutamyl-L-tryptophan (also referred to as SCV-07) as a vaccine enhancer. γ-D-glutamyl-L-tryptophan can be safely administered to human subjects without adverse effects. However, no clinical effect of this compound has been observed on oral mucositis raising doubt about the efficacy of this drug as an immunomodulator in humans.

A better understanding of the mechanism underlying the immunity-enhancing effect of the prior art compounds is needed to provide a basis for the selection of immunomodulatory compounds that are suitable to enhance the protective immunity elicited by an immunogen. In addition, a continued need exists to identify compounds that are suitable for enhancing the protective immunity elicited by a wide range of vaccines and, at the same time, can safely be administered to the general population including children and women of child-bearing age.

For instance, despite the use of potent adjuvants to modulate the immune response, some vaccines require repeated boost doses over an individual's lifespan in order to keep the immune system at a sufficiently high “threshold level” to counteract pathogen attack. A typical example is tetanus vaccination which has to be boosted every other year to keep anti-toxin antibody titres sufficiently high to guarantee protection. Furthermore, for some pathogens effective vaccines do not exist yet.

### SUMMARY OF THE INVENTION

The invention changes the focus away from boosting the adaptive immune response to include the innate immune response as an essential element of pathogen defence in any vaccination strategy. More specifically, the methods of the invention are aimed at synergising a pre-existing adaptive immune response against a given pathogen with the activation of a non-specific innate immune response by administering an immunomodulatory compound. This invention is designed to generate protective immunity for vaccines which by themselves are not fully protective. This includes vaccines that are prepared using a specific strain of a pathogen, but lack broad coverage due to the high genetic variability of circulating strains of this pathogen, or vaccines targeted at a pathogen that is able to evade immune responses by various mechanisms. Examples of vaccines that may benefit from the present invention include vaccines against influenza, Neisseria meningitidis serogroup B, Streptococcus pneumoniae, Staphylococcus aureus, Mycobacterium tuberculosis, Streptococcus pyogenes, HIV, and malaria. This invention provides a fast-acting protective immunity in vaccinated subjects which have high risk of being infected by the pathogen against which they have been previously vaccinated. This may include subjects who have close contact with infected patients, receive surgical operations, or have open wounds or severe burns as well as patients who are unable to effectively mount an immune response by themselves due to hemodialysis, immune depression, etc.

The invention is based on the inventors' surprising discovery that oral administration of nicotinamide (NAM), an immunomodulatory compound, subsequent to administration of a Staphylococcus aureus vaccine can enhance the protective immunity of mice against an otherwise lethal dose of S. aureus. This effect was completely unexpected.

The inventors believe that the effect is not limited to NAM, but can be achieved more generally by other immunomodulatory compounds. Similarly, it is the inventors' belief that the immunity-enhancing effect observed with the tested S. aureus vaccine can also be attained with other vaccines. Thus the invention includes immunomodulatory compounds and their use in enhancing the protective immunity elicited by an immunogen. In some embodiments, the immunomodulatory compounds in accordance with the invention do not include thalidomide and thalidomide derivatives such as lenalidomide and pomalidomide and optionally also exclude certain immunomodulatory peptides such as γ-D-glutamyl-L-tryptophan (see below).

In particular, the invention relates to the use of immunomodulatory compounds that stimulate the innate immune system as vaccine enhancers. Immunomodulatory compounds that stimulate the innate immune system are referred to hereinafter as “innate immunity stimulators”. More specifically, the invention relates to methods for enhancing the protective immunity elicited by an immunogen that comprise administering an immunomodulatory compound to a patient separately but simultaneously with, or prior or subsequent to, the administration of a vaccine which contains the immunogen. More preferably, the invention relates to a method for enhancing the protective immunity elicited by an immunogen that comprises separately administering an immunomodulatory compound such as an innate immunity stimulator to a patient subsequent to the administration of a vaccine which contains the immunogen.

Administering the immunomodulatory compound separately but simultaneously with, or prior or subsequent to, the administration of a vaccine against a pathogen may result in better protection against a subsequent challenge with said pathogen, in particular under circumstances where vaccination would normally not provide adequate protection. Better protection is achieved particularly when the immunomodulatory compound is administered subsequent to the administration of the vaccine.

Administration of an only weakly immunogenic vaccine, or a vaccine against a different but related pathogen, may result in adequate protection from infection due to the enhanced protective immune response elicited in a subject exposed to the vaccine before, after or during administration of the immunomodulatory compound. Preferably, the vaccine is administered first, followed by the administration of the immunomodulatory compound. These administration schemes may be particularly useful in cases such as an epidemic or pandemic caused by a pathogen for which no adequate or fully protective vaccine exists.

Alternatively, a reduced-dose vaccine (e.g., in the case of a vaccine shortage during a pandemic or in cases where production of large amounts of an immunogen is technically or economically not feasible) may be rendered more effective by administering an immunomnodulatory compound of the invention separately but simultaneously with, or prior or subsequent to, the administration of the reduced-dose vaccine. Effectiveness of a reduced-dose vaccine is especially enhanced if the immunomodulatory compound is administered subsequent to the administration of the reduced-dose vaccine. In addition, the immunomodulatory compound of the invention will render conventional vaccines that already provide good protective immunity more effective, e.g. by inducing higher antibody titres against the immunogens included in these vaccines, or by eliciting a more rapid immune response after vaccination.

Similarly, a polysaccharide-, peptide- or hapten-conjugate vaccine that yields only a weak immune response can be enhanced by administering to a patient the immunomnodulatory compound of the invention separately but simultaneously with, prior or subsequent to, the administration of the vaccine. This is particularly advantageous because it renders previously ineffective or only partially effective vaccines fully protective. To achieve this effect, the immunomodulatory compound is preferably administered after administration of the vaccine. Examples of vaccines that have failed to induce an adequate protective response in the majority of the tested subjects include vaccines to nicotine and cocaine as well as childhood vaccines to respiratory syncytial virus (RSV), and Staphylococcus aureus.

In one particular aspect of the invention, the immunomodulatory compound is administered to a patient who has previously been vaccinated with an antigen prior to a subsequent exposure to the antigen. For example, the patient may have previously been vaccinated against a pathogen that is usually associated with a hospital-acquired infection (e.g. S. aureus, in particular methicillin-resistant S. aureus [MRSA]). In such a case, the patient will be administered the immunomodulatory compound according to the invention prior to a hospital stay during which he or she might be exposed to the pathogen to enhance a previously established adaptive immune response by boosting the innate immune response. Similarly, a patient may have previously received a vaccine against prepandemic or pandemic influenza vaccine. During a pandemic, an immunomodulatory compound according to the invention will be administered to the patient to enhance a previously established adaptive immune response against an influenza antigen of or pandemic or pandemic influenza vaccine by boosting the innate immune response.

In some instances, administering the immunomodulatory compound of the invention before, during, or after administration of a vaccine may reduce the number of booster vaccinations needed. For example, with some vaccines, several boosters are needed after the priming vaccination to yield protective immunity against the vaccine antigen(s) in the majority of subjects to which the vaccine is administered. By administering the immunomodulatory compound of the invention separately but simultaneously with, or prior or subsequent to, the administration of the primer vaccine, the number of boosts needed to elicit protective immunity can be reduced. Administering the immunomodulatory compound after the primer vaccination is particularly effective in reducing the number of boosts needed to elicit protective immunity. For example, if two booster vaccinations are needed, the number can be reduced to one booster vaccination. In some cases, no booster vaccination will be needed.

To elicit a more effective immune response, one or more adjuvants are usually added to an immunogenic composition to boost the response to the immunogen. However, the presence of a traditional adjuvant in an immunogenic composition may deter some people from getting vaccinated. In some aspects of the invention, the immunomodulatory compound of the invention may be used to enhance the immune response to an immunogenic composition in place of a traditional adjuvant. In another aspect of the invention, the administration of an immunomodulatory compound in conjunction with the administration of an immunogenic composition (i.e. before, during or after administration of the immunogenic composition) may allow the reduction of the adjuvant dose that is usually present in the immunogenic composition. Thus, while the use of an adjuvant may not be completely avoided, the use of the immunomodulatory compound in conjunction with the vaccine may result in a more effective vaccination regimen (e.g. because the adjuvant dose and antigen dose can be further reduced without affecting the efficacy of the vaccine). Providing a more effective vaccination regimen with a vaccine comprising lower amounts of adjuvant and/or antigen may find greater acceptance, in particular among people who currently avoid getting vaccinated.

Innate immunity stimulators have been found to be particularly effective immunomodulatory compounds for enhancing the protective immunity elicited by a vaccine. Many innate immunity stimulators are known in the art and they can be identified by their capacity to modulate the innate immune response to a pathogen.

For example, a compound that enhances the expression of one or more genes encoding antimicrobial peptides may be considered an innate immunity stimulator. Such a compound may further modulate the expression of other effector molecules of the innate immune system. For instance, such a compound may additionally inhibit or induce the expression of inducible NO synthase (iNOS), reduce or increase interferon-γ-induced MHC class I expression, decrease or increase intracellular adhesion molecule 1 (ICAM-1) expression, and/or inhibit or induce the expression of IL-1β, IL-6, IL-8 and TNF-α.

In a specific embodiment, an innate immunity stimulator of the invention enhances the expression of genes encoding antimicrobial peptides. Preferably, such a compound simultaneously inhibits iNOS, reduces interferon-γ-induced MHC class I (in particular HLA-DR and -DP) expression, and/or decreases ICAM-1 expression. Additionally, such a compound may also inhibit the expression of IL-1β, IL-6, IL-8 and TNF-α. In a particular embodiment, an innate immunity stimulator of the invention combines all of these characteristics. An example for such an innate immunity stimulator is NAM.

In addition, an innate immunity stimulator, such as a TLR receptor agonist, may modulate the expression of cytokines that facilitate the recruitment and/or activation of professional phagocytes (e.g. neutrophils, monocytes, macrophages, dendritic cells). Examples of cytokines having these characteristics include IL-23, IL-22, IL-17, IL-13, IL-5, IL-4, IL-2, IL-1β, TNF-α, and INF-γ.

Without being bound by any particular theory, an innate immune stimulator in accordance with the invention may act by activating an innate immunity signaling pathway that is independent of the Nlrp3-inflammasome and requires the adaptor protein MyD88. The innate immune stimulator may signal through MyD88 to induce secretion of G-CSF and IL-5. G-CSF and IL-5 secretion can be measured by determining the serum levels of these cytokines. Animals exposed to the innate immune stimulator will have increased serum levels of G-SCF and IL-5 in comparison to serum levels of these cytokines in control animals that have not been exposed to the innate immune stimulator. Innate immune stimulators having these characteristics include MF59 and complete Freund's adjuvant.

In one aspect, the present invention relates to the use of nicotinamide or derivatives thereof in enhancing the protective immunity elicited by an immunogen. Specifically, the invention relates to methods for enhancing the protective immunity elicited by an immunogen that comprise administering nicotinamide or a derivative thereof to a patient separately but simultaneously with, or prior or subsequent to, the administration of a vaccine which contains the immunogen.

In a second aspect, the present invention relates to the use of benzamide or derivatives thereof in enhancing protective immunity elicited by an immunogen. Specifically, the invention relates to methods for enhancing the protective immunity elicited by an immunogen that comprise administering benzamide or a derivative thereof to a patient separately but simultaneously with, or prior or subsequent to, the administration of a vaccine which contains the immunogen.

In a third aspect, the present invention relates to the use of a TLR agonist in enhancing protective immunity elicited by an immunogen. Specifically, the invention relates to methods for enhancing the protective immunity elicited by an immunogen that comprise administering a TLR agonist to a patient separately but simultaneously with, or prior or subsequent to, the administration of a vaccine which contains the immunogen.

In a fourth aspect, the present invention relates to the use of an immunomodulatory compound in enhancing protective immunity elicited by an immunogen in a subject who has previously received an immunogenic composition containing the immunogen. Specifically, the invention relates to methods for enhancing the protective immunity elicited by an immunogen that comprise administering an immunomodulatory compound to a subject who has previously been vaccinated with a composition comprising the immunogen, wherein the immunomodulatory compound is administered during a 24-hour time period before and/or after a second exposure to the immunogen. For example, the immunomodulatory compound may be administered one day prior to the second exposure to the immunogen or one day before and one day after the second exposure. The second exposure is typically in the form of a live pathogen belonging to the same or related species from which the immunogen was derived.

In a specific embodiment, the invention relates to a method for immunising a subject, comprising administering to a subject (i) at least one immunogenic composition and (ii) an immunomodulatory compound other than lenalidomide and pomalidomide, wherein the immunomodulatory compound is administered to the subject for the first time more than 48 hours after administration of the immunogenic composition. Preferably, the immunomodulatory compound is an innate immunity stimulator such as NAM or a NAM derivative, benzamide or a benzamide derivative or a TLR agonist.

In another specific embodiment, the invention relates to the combined use of (i) at least one immunogenic composition and (ii) an immunomodulatory compound other than lenalidomide and pomalidomide in a method of immunising a subject, wherein the immunomodulatory compound is administered to the subject for the first time more than 48 hours after administration of the immunogenic composition. Thus, the invention also includes a combination of (i) at least one immunogenic composition and (ii) an immunomodulatory compound other than lenalidomide, and pomalidomide for separate or sequential administration, wherein the immunomodulatory compound is administered for the first time more than 48 hours after administration of the immunogenic composition. The invention further relates to a kit comprising (i) at least one immunogenic composition and (ii) an immunomodulatory compound in accordance with the invention. The invention also relates to a package comprising (i) at least one immunogenic composition and (ii) an information leaflet containing written instructions that an immunomodulatory compound may be administered to a subject for the first time more than 48 hours after the subject's receiving the immunogenic composition.

In a further specific embodiment, the invention relates to a method for immunising a subject, comprising administering to a subject (i) a first dose of an immunogenic composition as a prime, (ii) a second dose of the immunogenic composition as a boost, and (iii) an immunomodulatory compound, wherein administration of the first dose and the second dose are at least one month apart and administration of the immunomodulatory compound takes place between administration of the first and the second dose or after administration of the second dose.

In yet another specific embodiment, the invention relates to a method for immunising a subject, comprising administering to a subject (i) an immunogenic composition comprising S. aureus antigen and (ii) an immunomodulatory compound, wherein the immunomodulatory compound is administered to the subject at least 24 hours after administration of the immunogenic composition. Thus, the invention also includes an immunogenic composition comprising an S. aureus antigen and an immunomodulatory compound for combined use in a method of immunising a subject, wherein the immunomodulatory compound is administered to the subject at least 24 hours after administration of the immunogenic composition. The invention further relates to a combination of (i) an immunogenic composition comprising S. aureus antigen and (ii) an immunomodulatory compound for separate or sequential administration, wherein components (i) and (ii) are administered within 24 hours of each other. Furthermore, the invention relates to a kit comprising (i) an immunogenic composition comprising S. aureus antigen and (ii) an immunomodulatory compound. The invention also relates to a package comprising (i) an immunogenic composition comprising S. aureus antigen and (ii) an information leaflet containing written instructions that the immunomodulatory compound may be administered to a subject at least 24 hours after the subject's receiving the immunogenic composition.

In one specific embodiment, the invention relates to a method for immunising a subject, comprising administering to a subject (i) an immunogenic composition and (ii) nicotinamide, wherein nicotinamide is administered to the subject at least 24 hours after administration of the immunogenic composition. In another specific embodiment, the invention relates to the combined use of an immunogenic composition and nicotinamide in a method of immunising a subject, wherein the nicotinamide is administered to the subject at least 24 hours after administration of the immunogenic composition. Thus, the invention also includes a combination of (i) an immunogenic composition and (ii) nicotinamide for separate or sequential administration, wherein nicotinamide is administered to the subject at least 24 hours after administration of the immunogenic composition. The invention further relates to a kit comprising (i) an immunogenic composition and (ii) nicotinamide. The invention also relates to a package comprising (i) an immunogenic composition and (ii) an information leaflet containing written instructions that nicotinamide may be administered to a subject at least 24 hours after the subject's receiving the immunogenic composition.

In a further specific embodiment, the invention relates to a method for enhancing the protective immunity elicited by an immunogen, wherein the method comprises administering an immunomodulatory compound to a subject who has previously been vaccinated with a composition comprising the immunogen, wherein one or more doses of the immunomodulatory compound is administered during a 24-48-hour time period before and/or after a second exposure to the immunogen. Preferably, the second exposure is in the form of a live pathogen belonging to the same or a related species from which the immunogen was derived.

### DETAILED DESCRIPTION OF THE INVENTION

### The Immunomodulatory Compound

### Nicotinamide

The present invention is based on the discovery that oral administration of nicotinamide (NAM) subsequent to administration of an S. aureus vaccine can enhance the protective immunity of mice against an otherwise lethal dose of S. aureus.

NAM was originally identified to be an effective antimicrobial for use in treating infections with Mycobacterium tuberculosis [4]. Two structurally related compounds, pyrazinamide and isoniazid, were also found to have antimycobaterial activity. Combination therapy of nicotinamide and isoniazid in the treatment of pulmonary tuberculosis, however, proved ineffective and the use of NAM as antimycobacterial agent was subsequently abandoned [5].

Pozzilli et al. [6] compared the effect of BCG vaccination plus NAM treatment to NAM treatment alone in patients with newly diagnosed insulin-dependent diabetes mellitus (IDDM). Administration of the BCG vaccine had no additional therapeutic effect compared with NAM treatment alone.

WO2011/133692 [7] describes that NAM administered to mice daily beginning 24 hours prior to infection or 12 hours after infection with S. aureus dramatically enhances immune killing of the bacteria by increasing the activity of C/EBPε. Exposing bone-marrow derived macrophages from wild-type mice to NAM increased levels of lysine acetylation on core histone H3 in the promoter region of C/EBPε suggesting that NAM can act as an histone deacetylase (HDAC) inhibitor [7]. C/EBPε is a transcription factor specifically expressed in myeloid cells, and increased histone H3 acetylation of the C/EBPε promoter was associated with elevated C/EBPε mRNA and protein level as well as increased expression of downstream antimicrobials such as cathelicidin(-related) antimicrobial peptide (CAMP) and lactoferrin [7]. The same data are also subject of a scientific publication [8]. The observed effects of NAM on bacterial killing and clearance are different from the inventors' finding of an immunity-enhancing effect of NAM on an immune response elicited by the administration of a vaccine.

NAM displays potent anti-inflammatory properties and has been used in the treatment of a variety of inflammatory skin conditions [9]. In in vitro experiments using various cell types, NAM has been shown to inhibit iNOS, reduce interferon-γ-induced MHC class II (HLA-DR and -DP) expression, and decrease ICAM-1 expression. NAM also inhibits the expression of several proinflammatory cytokines, namely IL-1β, IL-6, IL-8 and TNF-α in a dose dependent manner, likely by acting on NF-κB [10,11].

The exact mechanism of action by which NAM exerts its multiple therapeutic effects has not been elucidated. It is the inventors' belief that both the NAM's immunomodulatory effect on the expression of several proinflammatory cytokines and its epigenetic effect on the expression of antimicrobials may play a role in its ability to enhance the protective immunity elicited by an immunogen. The inventors believe that NAM influences multiple effector cells involved in orchestrating the innate immune response and boosts the innate immune system after exposure to an antigen. The boost is believed to lead to a more effective innate immune response which may facilitate the development of a long-lasting adaptive immune response to an antigen or enhance a previously established adaptive immune response to an antigen upon subsequent exposure to the same antigen. This was unexpected in view of reports in the prior art literature showing that NAM supresses many proinflammatory pathways which are believed to be essential for an effective innate immune response, such as the induction of iNOS and the expression of ICAM-1 and various proinflammatory cytokines.

### Nicotinamide Derivatives

Nicotinamide-derived drugs such as pyrazinamide have an activity spectrum similar to that of nicotinamide (see reference 4) and therefore may also be useful compounds for enhancing the immune response to an immunogen. Nicotinamide derivatives with immunomodulatory activity are well-known in the art (see, e.g., references 12, 13 and 14). Thus, in one embodiment of the invention, the immunomodulatory compound is nicotinamide or a derivative thereof. Nicotinamide derivatives having PDE4-inhibitory activity are less preferred in the context of the present invention.

### Benzaminde and Benzamide Derivatives

As expected in view of their structural similarity to nicotinamide and its derivatives, benzamide and benzamide derivatives exhibit immunomodulatory activities similar to those observed with nicotinamide (see [11]). Particularly useful are N-substituted benzamides which have been shown to inhibit NF-κB [15,16]. Thus, in a further embodiment of the invention, the immunomodulatory compound is benzamide or a derivative thereof. The addition of an aromatic N-acetyl group may enhance the immunomodulatory activity of benzamide and benzamide derivatives such as procainamide. Benzamide derivates that induce apoptosis in cultured cells (e.g. the murine pre-B lymphocyte cell line 70Z/3) are less preferred.

The immunomodulatory compounds according to the invention can therefore be described in more general terms. The immunomodulatory compound of the invention is able to enhance the protective immunity elicited by an immunogen when administered to a subject separately but simultaneously with, or prior or subsequent to, the administration of the immunogen. The immunomodulatory compound of the invention may achieve this effect by inhibiting in cells of the immune system the expression of proinflammatory cytokines, e.g. IL-1β, IL-6, IL-8 and TNF-α Inhibition may be mediated by NF-κB. Thus, in a preferred embodiment of the invention, the immunomodulatory compound is an NF-κB inhibitor. Experimental systems for determining the immunomodulatory effect of a compound are well established in the art. For example, cytokine microarrays, PCR arrays or multiplex-bead ELISAs that measure e.g. IL-1β, IL-6, IL-8 and TNF-α expression or NF-κB activation assays that quantitate the nuclear import of NF-κB can be used to determine the response of a mammalian cell after exposure to an immunomodulatory compound. Preferably, the immunomodulatory compound of the invention is selected from the group of compounds comprising nicotinamide or derivatives thereof and benzamide or derivatives thereof. In particular, an immunomodulatory compound according to the invention is a compound of formula I:

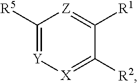

or a pharmaceutically acceptable salt thereof, wherein:

X is selected from N and CR³;

Y is selected from N and CR⁴;

Z is selected from N and CR⁶;

R¹ is selected from C(O)NR⁷R⁸, NR⁷R⁸ and NR⁷C(O)R⁸;
each of R², R³, R⁴, R⁵, R⁶, R⁷ and R⁸ is independently selected from hydrogen, hydroxyl, cyano, nitro, C₁₋₆-alkyl, C₂₋₆-alkenyl, C₂₋₆-alkynyl, C₁₋₆-alkoxy, C₂₋₆-alkenyloxy, C₂₋₆-alkynyloxy, halogen, C₁₋₆-alkylcarbonyl, carboxy, C₁₋₆-alkoxycarbonyl, amino, C₁₋₆-alkylamino, di-C₁₋₆-alkylamino, C₁₋₆-alkylaminocarbonyl, di-C₁₋₆-alkylaminocarbonyl, C₁₋₆-alkylcarbonylamino, C₁₋₆-alkylcarbonyl(C₁₋₆-alkyl)amino, C₁₋₆-alkylsulfonylamino, C₁₋₆-alkylsulfonyl(C₁₋₆-alkyl)amino, C₁₋₆-thioalkyl, C₁₋₆-alkylsulfinyl, C₁₋₆-alkylsulfonyl, aminosulfonyl, C₁₋₆-alkylaminosulfonyl and di-C₁₋₆-alkylaminosulfonyl, optionally wherein each of the aforementioned hydrocarbon groups is substituted by one or more halogen, hydroxyl, C₁₋₆-alkoxy, amino, C₁₋₆-alkylamino, and di-C₁₋₆-alkylamino or cyano.

Cycloalkyl or cycloalkylene represents a 3 to 14-membered monocyclic or bicyclic carbocyclic ring, wherein the monocyclic ring or one of the bicyclic rings is saturated or partially unsaturated and may optionally further comprise a —C(O)— ring member, and the other ring may be aromatic, saturated or partially unsaturated and may include one to three ring members selected from —C(O)—, —N(R¹⁹)q-, —O— and S(O)r, where R¹⁹ is H or C₁₋₆-alkyl, q is 0-1 and r is 0-2;

Aryl or arylene represents a 6 to 14-membered monocyclic or bicyclic carbocyclic ring, wherein the monocyclic ring or one of the bicyclic rings is aromatic and the other ring may be aromatic, saturated or partially unsaturated and may include one to three ring members selected from —C(O)—, —N(R²⁰)q-, —O— and —S(O)r-, where R²⁰ is H or C₁₋₆-alkyl, q is 0-1 and r is 0-2;

Heteroaryl or heteroarylene represents a 5 to 14-membered monocyclic or bicyclic ring, wherein the monocyclic ring or one of the bicyclic rings is an aromatic group comprising either (a) 1-4 nitrogen atoms, (b) one oxygen or one sulphur atom or (c) one oxygen atom or one sulphur atom and 1 or 2 nitrogen atoms, and the other ring may be aromatic, saturated or partially unsaturated, and may include one to three ring members selected from —C(O)—, —N(R²¹)q-, —O— and —S(O)r-, where R²¹ is H or C₁₋₆-alkyl, q is 0-1 and r is 0-2; and Heterocycloalkyl or heterocycloalkylene represents a 3 to 14-membered monocyclic or bicyclic ring, wherein the monocyclic ring or one of the bicyclic rings is a saturated or partially unsaturated group comprising one or two ring members selected from —N(R²²)—, —O— and —S(O)r- and may optionally further comprise a —C(O)— ring member, and the other ring may be aromatic, saturated or partially unsaturated, and may include one to three ring members selected from —C(O)—, —N(R²³)q-, —O— and —S(O)r-, where R²² or R²³ is H or C₁₋₆-alkyl, q is 0-1 and r is 0-2.

Specific compounds falling within the scope of formula I are disclosed in references 11, 12, 15, 16 and 17.

Preferably, a compound falling within the scope of formula I is able to inhibit the activity of NF-κB in a mammalian cell in vivo (see [17]). Whether such a compound has in vivo NF-κB inhibitory activity can be tested or confirmed in vitro using cultured mammalian cells, typically by measuring LPS-induced TNF-α production as surrogate marker. Thus, in a preferred embodiment, the invention relates to an NF-κB inhibitor selected from the group of compounds described by formula I.

A preferred subgroup of the immunomodulatory compounds of the invention are those described by formula II:

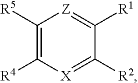

or a pharmaceutically acceptable salt thereof; wherein

X is selected from N and CR³;

Z is selected from N and CR⁶;

R¹ is selected from C(O)NR⁷R⁸ or NR⁷C(O)R⁸;

R², R³, R⁴, R⁵ and R⁶ are independently selected from amino, halogen, hydrogen, amide, alkyl, and alkoxy;

R⁷ is hydrogen;

R⁸ is

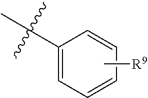

or C₁₋₆—NR¹⁰R¹¹;

R⁹ is C₁₋₆-alkyl optionally substituted with one or more selected from halogen or hydroxyl; and

R¹⁰ and R¹¹ are independently C₁₋₆-alkyl.

Specific examples of immunomodulatory compounds of the invention include the following molecules:

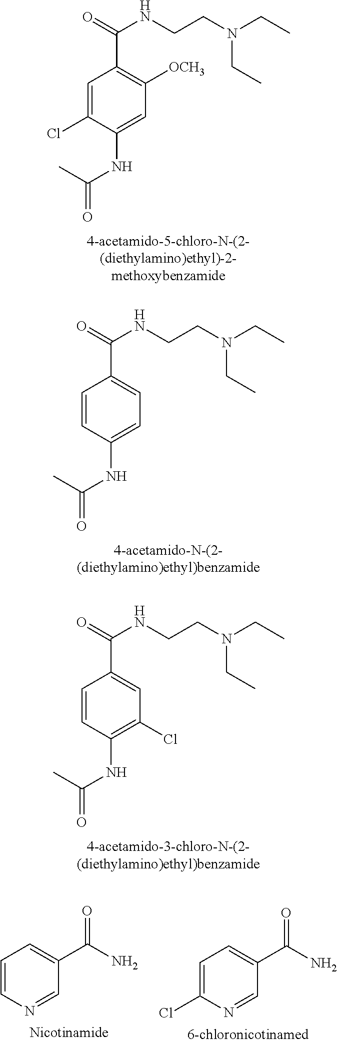

Bone marrow toxicity is an adverse effect frequently observed in patients receiving lenalidomide or pomalidomide. In addition, both drugs are potentially teratogenic in humans. Thus, even though lenalidomide and pomalidomide have been shown to enhance the immune response to vaccines, the risks associated with the administration of these compounds far outweigh the expected benefits and make them unsuitable for general application as vaccine enhancers. In contrast, no significant side effects are associated with NAM treatment of human subjects [18]. NAM is neither oncogenic nor teratogenic in humans. Only at very high doses (>3 g/day), NAM has been reported to be reversible hepatotoxic in animals and humans. Hence, in view of NAM's excellent safety profile, the present invention makes it feasible for the first time to use an immunomodulatory compound to enhance the immune response to a vaccine in children, the elderly and women of child-bearing age. Therefore NAM is a preferred immunomodulatory compound of the invention.

Preferred compounds falling within the scope of formula I will have pharmacological characteristics similar to NAM, e.g. they will have very little to no toxicity, will not be cytotoxic, will not be teratogenic in humans, have no oncogenic properties etc.

In some embodiments of the invention, immunomodulatory compounds do not include thalidomide and any of its derivatives as described in reference 1 (which is incorporated herewith by reference). In specific embodiments of the invention, immunomodulatory compounds do not include 1-oxo- and 1,3-dioxo-2-(2,6-dioxopiperidin-3-yl) isoindolines substituted with amino in the benzo ring which have the following structure:

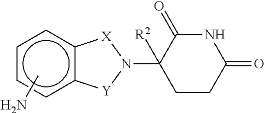

in which one of X and Y is C═O, the other of X and Y is C═O or CH₂, and R² is hydrogen or lower alkyl, in particular methyl.

In other embodiments of the invention, immunomodulatory compounds do not include immunomodulatory peptides of the following structure:

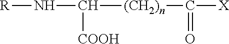

in which n is 1 or 2, R is hydrogen, acyl, alkyl or a peptide fragment, and X is an aromatic or heterocyclic amino acid (e.g. L-tryptophan or D-tryptophan) or a derivative thereof, optionally wherein the carbon of the CH group shown in has a stereoconfiguration, when n is 2, that is different from the stereoconfiguration of X. Derivatives of the aromatic or heterocyclic amino acids for X include amides, mono- or di-(C₁-C₆) alkyl substituted amides, arylamides, and (C₁-C₆) alkyl or aryl esters. Acyl or alkyl moieties for R include branched or unbranched alkyl groups of 1 to 6 carbons, acyl groups from 2 to 10 carbon atoms, and blocking groups such as carbobenzyloxy and t-butyloxycarbonyl. In specific embodiments of the invention, the immunomodulatory compound is not γ-D-glutamyl-L-tryptophan.

### TLR Agonists

The inventors believe that the immune response-enhancing effect observed with NAM can also be achieved by other immunomodulatory compounds that act on the cells of the innate immune system.

NAM treatment stimulates the innate immune response by increasing the expression of antimicrobial peptides such as cathelicidin-related antimicrobial peptide [7]. The inventors believe that immunomodulatory compounds that increase the expression of cathelicidin-related antimicrobial peptide may also enhance the protective immune response elicited in a subject exposed to the vaccine when administered before, after or during vaccination. Toll-like receptor (TLR) agonists have been shown to induce expression of cathelicidin-related antimicrobial peptide hCAP-18/LL-37 in human cells [19]. Thus, in one aspect of the invention, TLR agonists are suitable immunomodulatory compounds for practising the methods of the invention.

The use of TLR agonists to enhance the response to a vaccine has been described in the prior art. For example, Nava-Parada et al. [20] tested what influence the administration of a TLR9 agonist (ODN-1826) had on the effectiveness of a peptide vaccine for the treatment and prevention of spontaneous breast tumours. The TLR9 agonist was administered subcutaneously in five daily injections at days −2, −1, 0, 1, 2 (day 0: vaccination). Nava-Parada et al. suggest that CpG serves a critical role in generating an effective cytotoxic T-cell response against a tumour. Interestingly, the anti-tumour effect was observed when CpG was administered in five daily injections without the peptide vaccine.

Zheng et al. [21] report that paired but not solitary combinations of TLR3 agonist (polyI-C) and a TLR9 agonist (ODN-1826) stimulated IL-12 secretion from dendritic cells in vitro and synergized with vaccination of dendritic cell MCA205 fibrosarcoma electrofusion hybrids to achieve potent rejection of established MCA205 sarcomas in syngeneic mice. The TLR agonists were administered at days 0, 3, 7 (day 0: vaccination). Zheng et al. suggest that IL-12 plays a significant role in the adjuvant properties of paired TRL agonists.

Taillardet et al. [22] disclose that TLR agonists (and in particular the TLR9 agonist CpG1668) allow generation of long-lasting antipneumococcal humoral immunity in response to a plain polysaccharide vaccine, provided that their administration is delayed until the second day after vaccination. Taillardet et al. postulate that CpG1668 protects B-cells from activation induced cell death possibly favoured by the extensive BCR cross-linking promoted by thymus-independent antigens such as the tested polysaccharide vaccine.

Jensen et al. [23] report that immunization with a pneumococcal vaccine followed by administration of a TLR9 agonist 48 hours later significantly improved nasopharyngeal protection against pneumococcal infection when compared to simultaneous administration. Jensen et al. observed that Th1 and Th2 responses were blocked by the simultaneous administration of the pneumococcal vaccine and the TLR9 agonist but were partly restored when administration of the TLR9 agonist was delayed by 48 hours.

Without being bound by any particular theory, the inventors believe that the mechanism of action underlying the present inventions differs from the mechanism that was thought to give rise to the observation reported in references 20-23. In particular, the inventors believe that the immunomodulatory compounds according to the invention exert their immunomodulatory effect by negatively affecting the expression of proinflammatory cytokines and positively influencing the expression of antimicrobial factors. For example, macrophages that have been stimulated for a prolonged period of time (≧24 hours) with a TLR agonist such as lipopolysaccharide (LPS) have been shown to become tolerant to a TLR agonist's stimulating activity [24, 25]. In particular, genes encoding proinflammatory cytokines are silenced and do no longer respond to TLR signalling. In contrast, genes encoding antimicrobial peptides are non-tolerizeable and remain inducible by TLR signaling. Thus, exposure to NAM and prolonged stimulation with a TLR agonist have similar effects on gene expression in cells of the innate immune system.

In accordance with the invention, a TLR agonist preferably is an agonist of a human TLR. The TLR agonist can activate any of TLR1, TLR2, TLR3, TLR4, TLR5, TLR6, TLR7, TLR8, TLR9 or TLR11; preferably it can activate human TLR2, human TLR7, human TLR8 or human TLR9. In some aspects of the invention, the immunomodulatory compound is not poly I:C. In some instances, the immunomodulatory compound is not a TLR3 agonist. In other aspects of the invention, the immunomodulatory compound is not ODN-1826 or CpG1668. In some instances, the immunomodulatory compound is not a TLR9 agonist.

Agonist activity of a compound against any particular Toll-like receptor can be determined by standard assays. Companies such as Imgenex and Invivogen supply cell lines which are stably co-transfected with human TLR genes and NFκB, plus suitable reporter genes, for measuring TLR activation pathways. They are designed for sensitivity, broad working range dynamics and can be used for high-throughput screening. Constitutive expression of one or two specific TLRs is typical in such cell lines. See also reference 26. Many TLR agonists are known in the art e.g. reference 27 describes certain lipopeptide molecules that are TLR2 agonists, references 28 to 31 each describe classes of small molecule agonists of TLR7, and references 32 & 33 describe TLR7 and TLR8 agonists for treatment of diseases.

Useful TLR agonists include any of the following compounds, as disclosed in references 27-69.

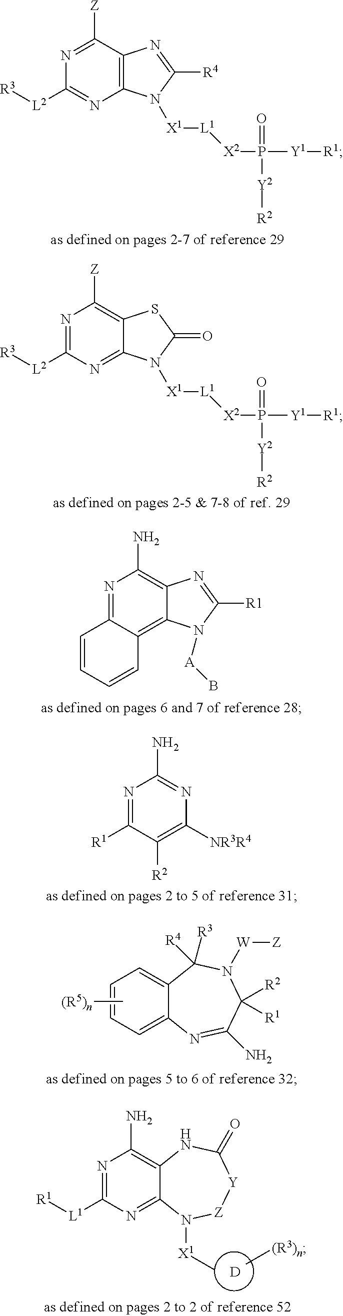

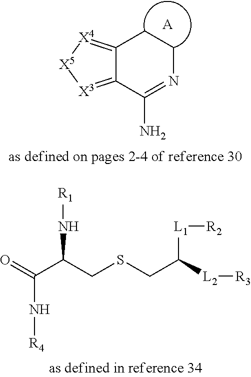

A TLR agonist used with the invention may include at least one adsorptive moiety. The inclusion of such moieties in TLR agonists allows them to adsorb to insoluble aluminium salts (e.g. by ligand exchange or any other suitable mechanism) and improves their immunological behaviour [35]. Phosphorus-containing adsorptive moieties are particularly useful, and so an adsorptive moiety may comprise a phosphate, a phosphonate, a phosphinate, a phosphonite, a phosphinite, etc.

Preferably the TLR agonist includes at least one phosphonate group.

Thus, in preferred embodiments, a composition of the invention includes a TLR7 agonist which includes a phosphonate group. This phosphonate group can allow adsorption of the agonist to an insoluble aluminium salt [35].

TLR agonists useful with the invention may include a single adsorptive moiety, or may include more than one e.g. between 2 and 15 adsorptive moieties. Typically a compound will include 1, 2 or 3 adsorptive moieties.

Phosphorus-containing TLR agonists useful with the invention can be represented by formula (A1):

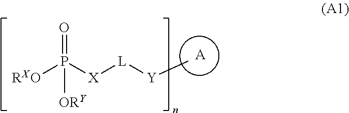

wherein:
   R^{X} and R^{Y} are independently selected from H and C₁-C₆ alkyl;
   X is selected from a covalent bond, 0 and NH;
   Y is selected from a covalent bond, 0, C(O), S and NH;
   L is a linker e.g. selected from, C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
   each p is independently selected from 1, 2, 3, 4, 5 and 6;
   q is selected from 1, 2, 3 and 4;
   n is selected from 1, 2 and 3; and
   A is a TLR agonist moiety.

In some embodiments, the TLR agonist moiety ‘A’ has a molecular weight of less than 1000 Da. In some embodiments, the TLR agonist of formula (A1) has a molecular weight of less than 1000 Da.

In one embodiment, the TLR agonist according to formula (A1) is as follows: R^{X} and R^{Y} are H; X is O; L is selected from C₁-C₆ alkylene and —((CH₂)ₚO)_{q}(CH₂)ₚ— each optionally substituted with 1 to 2 halogen atoms; p is selected from 1, 2 and 3; q is selected from 1 and 2; and n is 1. Thus in these embodiments the adsorptive moiety comprises a phosphate group.

In other embodiments, the TLR agonist according to formula (A1) is as follows: R^{X} and R^{Y} are H; X is a covalent bond; L is selected from C₁-C₆ alkylene and —((CH₂)ₚO)_{q}(CH₂)ₚ— each optionally substituted with 1 to 2 halogen atoms; p is selected from 1, 2 or 3; q is selected from 1 or 2; and n is 1. Thus in these embodiments the adsorptive moiety comprises a phosphonate group.

Further phosphonate group-containing TLR agonists which can be adsorbed to insoluble metal salts are described in reference 35.

Preferred TLR agonists are water-soluble. Thus they can form a homogenous solution when mixed in an aqueous buffer with water at pH 7 at 25° C. and 1 atmosphere pressure to give a solution which has a concentration of at least 50 μg/ml. The term “water-soluble” thus excludes substances that are only sparingly soluble under these conditions.

Useful TLR agonists for use alone or as moiety ‘A’ in formula A1 include those described in reference 35 having formula (C), (D), (E), (F), (G), (H), (I), (II), (J) or (K). Other useful TLR agonists are compounds 1 to 102 as defined in reference 35. The TLR7 agonists of references 28-31 and 36-52 and the TLR8 agonists of references 32 & 33 are also useful in practising the invention. Preferred TLR7 agonists have formula (K), such as compound K2 identified below. These can be used as salts e.g. the arginine salt of K2.

Preferred TLR4 agonists are analogs of monophosphoryl lipid A (MPL), as described in more detail below. For instance, a useful TLR4 agonist is a 3d-MPL.

A composition of the invention can include more than one TLR agonist. These two TLR agonists are different from each other and they can target the same TLR or different TLRs. Both agonists can be adsorbed to an aluminium salt.

### TLR4 Agonists

Compositions of the invention can include a TLR4 agonist, and most preferably an agonist of human TLR4. TLR4 is expressed by cells of the innate immune system, including conventional dendritic cells and macrophages [53]. Triggering via TLR4 induces a signalling cascade that utilizes both the MyD88- and TRIF-dependent pathways, leading to NF-κB and IRF3/7 activation, respectively. TLR4 activation typically induces robust IL-12p70 production and strongly enhances Th1-type cellular and humoral immune responses.

Various useful TLR4 agonists are known in the art, many of which are analogs of endotoxin or lipopolysaccharide (LPS). For instance, the TLR4 agonist can be:
(i) 3d-MPL (i.e. 3-O-deacylated monophosphoryl lipid A; also known as 3-de-O-acylated monophosphoryl lipid A or 3-O-desacyl-4′-monophosphoryl lipid A). This derivative of the monophosphoryl lipid A portion of endotoxin has a de-acylated position 3 of the reducing end of glucosamine. It has been prepared from a heptoseless mutant of Salmonella minnesota, and is chemically similar to lipid A but lacks an acid-labile phosphoryl group and a base-labile acyl group. Preparation of 3d-MPL was originally described in ref 54, and the product has been manufactured and sold by Corixa Corporation. It is present in GSK's ‘AS04’ adjuvant. Further details can be found in references 55 to 58.
(ii) glucopyranosyl lipid A (GLA) [59] or its ammonium salt:

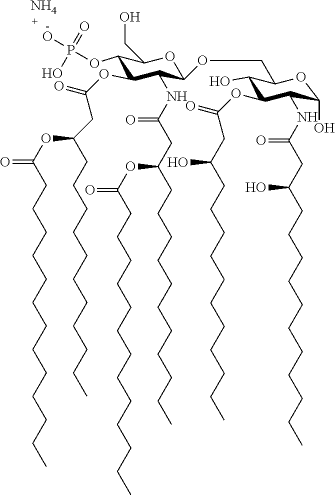

(iii) an aminoalkyl glucosaminide phosphate, such as RC-529 or CRX-524 [60-62]. RC-529 and CRX-524 have the following structure, differing by their R₂ groups:

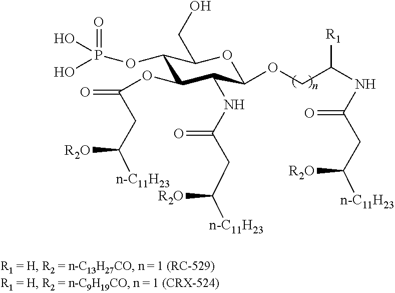

(iv) compounds containing lipids linked to a phosphate-containing acyclic backbone, such as the TLR4 antagonist E5564 [63,64]:

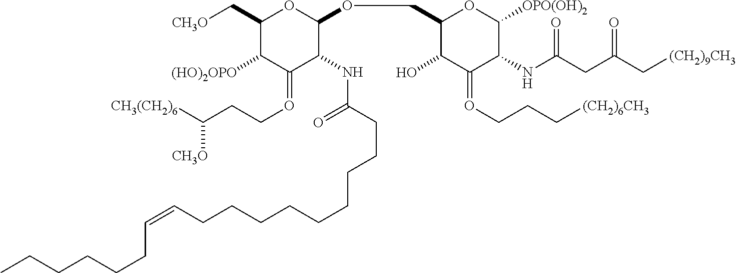

(v) A compound of formula I, II or III as defined in reference 65, or a salt thereof, such as compounds ‘ER 803058’, ‘ER 803732’, ‘ER 804053’, ‘ER 804058’, ‘ER 804059’, ‘ER 804442’, ‘ER 804680’, ‘ER 803022’, ‘ER 804764’ or ‘ER 804057’. ER 804057 is also known as E6020 and it has the following structure:

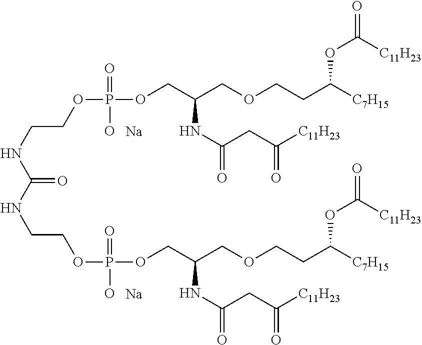

whereas ER 803022 has the following structure:

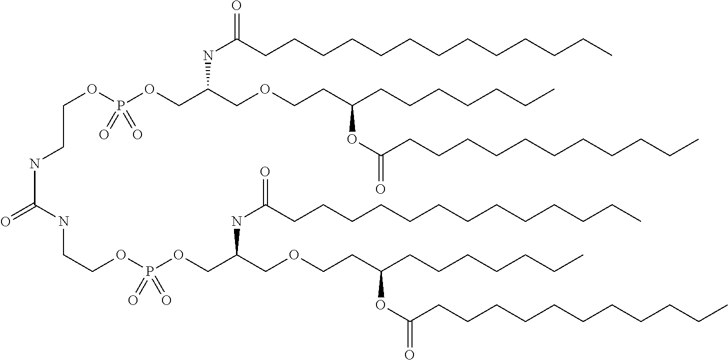

(vi) One of the polypeptide ligands disclosed in reference 66.

Any of these TLR4 agonists can be used with the invention.

A composition of the invention can include an aluminium salt to which the TLR4 agonist is adsorbed. TLR4 agonists with adsorptive properties typically include a phosphorus-containing moiety which can undergo ligand exchange with surface groups on an aluminium salt, and particularly with a salt having surface hydroxide groups. Thus a useful TLR4 agonist may include a phosphate, a phosphonate, a phosphinate, a phosphonite, a phosphinite, a phosphate, etc. Preferred TLR4 agonists include at least one phosphate group [35] e.g. the agonists (i) to (v) listed above.

The preferred TLR4 agonist for use with the invention is 3d-MPL. This can be adsorbed to an aluminium phosphate adjuvant, to an aluminium hydroxide adjuvant, or to a mixture of both [67].

3d-MPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R^{2′}.

The group attached to carbon 3′ has formula —O—CO—CH₂—CR³R^{3′}. A representative structure is:

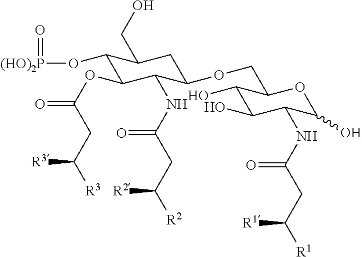

Groups R¹, R² and R³ are each independently —(CH₂)ₙ—CH₃. The value of n is preferably between 8 and 16, more preferably between 9 and 12, and is most preferably 10.

Groups R^{1′}, R^{2′} and R^{3′} can each independently be: (a) —H; (b) —OH; or (c) —O—CO—R⁴, where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are —H then the 3d-MPL has only 3 acyl chains (one on each of positions 2, 2′ and 3′). When only two of R^{1′}, R^{2′} and R^{3′} are —H then the 3d-MPL can have 4 acyl chains. When only one of R^{1′}, R^{2′} and R^{3′} is —H then the 3d-MPL can have 5 acyl chains. When none of R^{1′}, R^{2′} and R^{3′} is —H then the 3d-MPL can have 6 acyl chains. The 3d-MPL used according to the invention can be a mixture of these forms, with from 3 to 6 acyl chains, but it is preferred to include 3d-MPL with 6 acyl chains in the mixture, and in particular to ensure that the 6 acyl chain form makes up at least 10% by weight of the total 3d-MPL e.g. ≧20%, ≧30%, ≧40%, ≧50% or more. 3d-MPL with 6 acyl chains has been found to be the most adjuvant-active form.

Thus the most preferred form of 3d-MPL for use with the invention is:

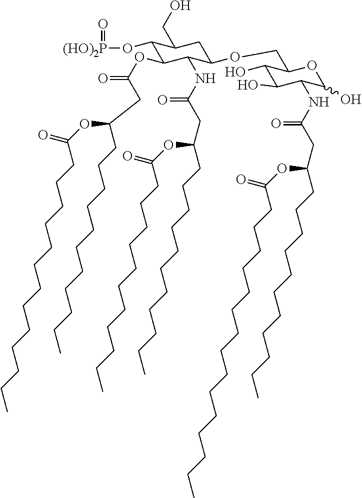

Where 3d-MPL is used in the form of a mixture then references to amounts or concentrations of 3d-MPL in compositions of the invention refer to the combined 3d-MPL species in the mixture. Typical compositions include 3d-MPL at a concentration of between 25 μg/ml and 200 μg/ml e.g. in the range 50-150 μg/ml, 75-125 μg/ml, 90-110 μg/ml, or about 100 μg/ml. It is usual to administer between 25-75 μg of 3d-MPL per dose e.g. between 45-55 μg, or about 50 μg 3d-MPL per dose.

In aqueous conditions, 3d-MPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3d-MPL) are preferred for use according to the invention because of their superior activity [68]. Preferred particles have a mean diameter less than 150 nm, more preferably less than 120 nm, and can even have a mean diameter less than 100 nm. In most cases, however, the mean diameter will not be lower than 50 nm. Where 3d-MPL is adsorbed to an aluminum salt then it may not be possible to measure the 3D-MPL particle size directly, but particle size can be measured before adsorption takes place. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.

Formula (K) [69]

The TLR agonist can be a compound according to formula (K):

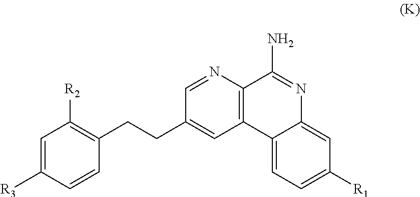

wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;
each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;
L⁴ is arylene or heteroarylene;
R² is H or C₁-C₆alkyl;
R³ is selected from C₁-C₄alkyl, -L³R⁵, -L¹R⁵, -L³R⁷, -L³L⁴L³R⁷, -L³L⁴R⁵, -L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁰;
R⁷ is —CF₂P(O)(OR⁹)₂ or —C(O)OR¹⁰;
R⁸ is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4.

They are described in reference 69 as TLR7 agonists. The compound of formula (K) is preferably of formula (K′):

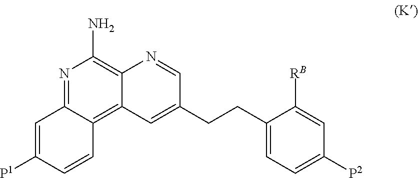

wherein:
P¹ is selected from H, C₁-C₆alkyl optionally substituted with COOH and —Y-L-X—P(O)(OR^{X})(OR^{Y});
P² is selected from H, C₁-C₆alkyl, C₁-C₆alkoxy and —Y-L-X—P(O)(OR^{X})(OR^{Y});
with the proviso that at least one of P¹ and P² is —Y-L-X—P(O)(OR^{X})(OR^{Y});
R^{B} is selected from H and C₁-C₆alkyl;
R^{X} and R^{Y} are independently selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6; and
q is selected from 1, 2, 3 and 4.

In some embodiments of formula (K′): P¹ is selected from C₁-C₆alkyl optionally substituted with COOH and —Y-L-X—P(O)(OR^{X})(OR^{Y}); P² is selected from C₁-C₆alkoxy and —Y-L-X—P(O)(OR′)(OR^{X})(OR^{Y}); R^{B} is C₁-C₆alkyl; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is selected from 1 and 2.

A specific embodiment of formula (K′) is compound “K2”, which can be described as 3-(5-amino-2-(2-methyl-4-(2-(2-(2-phosphonoethoxyl)ethoxy)ethoxy)phenethyObenzo [1]-[1,7]naphthyridin-8-yl)propanoic acid and has the following structure:

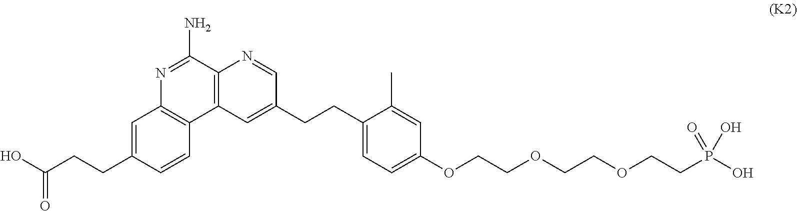 [SMILES: Cc1cc(OCCOCCOCCP(=O)(O)O)ccc1CCc1cnc2c(N)nc3cc(CCC(=O)O)ccc3c2c1]

### Formulation

The immunomodulatory compounds employed in practising the present invention can be administered by oral or parenteral routes, including intravenous, intramuscular, intraperitoneal, subcutaneous, transdermal, airway (aerosol), rectal, vaginal and topical (including buccal and sublingual) administration. In some aspects of the invention, oral administration is preferred.

For oral administration, the compounds of the invention will generally be provided in the form of tablets or capsules, as a powder or granules, or as an aqueous solution or suspension.

Tablets for oral use may include the immunomodulatory compound of the invention mixed with pharmaceutically acceptable excipients such as inert diluents, disintegrating agents, binding agents, lubricating agents, sweetening agents, flavouring agents, colouring agents and preservatives.

Suitable inert diluents include sodium and calcium carbonate, sodium and calcium phosphate, and lactose, while cornstarch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the immunomodulatory compound is mixed with a solid diluent, and soft gelatin capsules wherein the immunomodulatory compound is mixed with water or an oil such as peanut oil, liquid paraffin or olive oil.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the immunomodulatory compounds such carriers as are known in the art to be appropriate.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The compounds of the invention may also be presented as liposome formulations.

### Dosing

In general a suitable dose will be in the range of 0.1 to 300 mg per kilogram body weight of the subject per day. A preferred lower dose is 0.5 mg per kilogram body weight of the subject per day, a more preferred lower dose is 10 mg per kilogram body weight of subject per day, an even more preferred lower dose is 20 mg per kilogram body weight per subject per day. A suitable dose is preferably in the range of 6 to 150 mg per kilogram body weight per day, and most preferably in the range of 15 to 100 mg per kilogram body weight per day. The desired dose is preferably presented as two, three, four, five or six or more sub-doses administered at appropriate intervals throughout the day. These sub-doses may be administered in unit dosage forms, for example, containing 10 to 1500 mg, preferably 20 to 1000 mg, and most preferably 50 to 700 mg of active ingredient per unit dosage form.

For example, a suitable dose for NAM may be in the range of 1 to 100 mg per kilogram body weight of the subject per day. A preferred lower dose is 5 mg per kilogram body weight of the subject per day, a more preferred lower dose is 15 mg per kilogram body weight of subject per day, an even more preferred lower dose is 20 mg per kilogram body weight per subject per day. A suitable dose is in the range of 5 to 50 mg per kilogram body weight per day, preferably in the range of 10 to 40 mg per kilogram body weight per day. The desired dose is preferably presented as two, three, four, five or six or more sub-doses administered at appropriate intervals throughout the day. These sub-doses may be administered in unit dosage forms, for example, containing 250 to 1500 mg, preferably 500, 750 or 1000 mg of NAM per unit dosage form.

Immunogenic composition

Immunogenic compositions of the invention for administration to subjects are preferably vaccine compositions. Vaccines according to the invention may either be prophylactic (i.e. to prevent infection) or therapeutic (i.e. to treat infection), but will typically be prophylactic. Immunogenic compositions used as vaccines comprise an immunologically effective amount of immnogen(s)/antigen(s), as well as any other components, as needed. The antigen is preferably a component of a pathogen (e.g. a protein or polysaccharide) against which an immune response is to be elicited by the vaccine. However, in some instances, the immunogen may comprise virus particles or bacteria, in which case the virus particle or bacteria are preferably either inactivated or attenuated. In some embodiments, the immunogenic composition does not include Bacillus Calmette-Guérin (BCG) as immunogen. In other embodiments, the immunogenic composition does not contain live bacteria. More preferably, the immunogenic composition is not a live vaccine. Typically, the only immunogen(s)/antigen(s) included in an immunogenic composition according to the invention are those which are foreign to the organism to which the immunogenic composition is administered.

By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other rel-evant factors. It is expected that the amount will fall in a relatively broad range that can be determined through routine trials. The antigen content of compositions of the invention will generally be expressed in terms of the amount of protein or the amount of saccharide per dose.

The immunogenic composition will typically include a pharmaceutically acceptable carrier, and a thorough discussion of such carriers is available in reference 70.

The pH of the immunogenic composition is usually between 6 and 8, and more preferably between 6.5 and 7.5 (e.g. about 7). A stable pH in immunogenic compositions of the invention may be maintained by the use of a buffer e.g. a Tris buffer, a citrate buffer, phosphate buffer, or a histidine buffer. Thus immunogenic compositions of the invention will generally include a buffer.

The immunogenic composition may be sterile and/or pyrogen-free. The immunogenic composition may be isotonic with respect to humans.

Immunogenic compositions may include an immunological adjuvant. Thus, for example, they may include an aluminium salt adjuvant or an oil-in-water emulsion (e.g. a squalene-in-water emulsion). Suitable aluminium salts include hydroxides (e.g. oxyhydroxides), phosphates (e.g. hydroxyphosphates, orthophosphates), (e.g. see chapters 8 & 9 of reference 71), or mixtures thereof. The salts can take any suitable form (e.g. gel, crystalline, amorphous, etc.), with adsorption of antigen to the salt being preferred. The concentration of Al⁺⁺⁺ in a composition for administration to a subject is preferably less than 5 mg/ml e.g. ≦4 mg/ml, ≦3 mg/ml, ≦2 mg/ml, ≦1 mg/ml, etc. A preferred range is between 0.3 and 1 mg/ml. A maximum of 0.85 mg/dose is preferred. Aluminium hydroxide adjuvants are particularly suitable for use with meningococcal vaccines.

Infectious agents affect various areas of the body and so the immunogenic compositions of the invention may be prepared in various liquid forms. For example, the immunogenic compositions may be prepared as injectables, either as solutions or suspensions. The immunogenic composition may be prepared for pulmonary administration e.g. by an inhaler, using a fine spray. The composition may be prepared for nasal, aural or ocular administration e.g. as spray or drops, and intranasal vesicle vaccines are known in the art. Injectables for intramuscular administration are typical. Injection may be via a needle (e.g. a hypodermic needle), but needle-free injection may alternatively be used.

Immunogenic compositions may include an antimicrobial, particularly when packaged in multiple dose format. Antimicrobials such as thiomersal and 2-phenoxyethanol are commonly found in vaccines, but it is preferred to use either a mercury-free preservative or no preservative at all.

Immunogenic compositions may comprise detergent e.g. a Tween (polysorbate), such as Tween 80. Detergents are generally present at low levels e.g. <0.01%.

Immunogenic compositions may include sodium salts (e.g. sodium chloride) e.g. for controlling tonicity. A concentration of 10+2 mg/ml NaCl is typical e.g. about 9 mg/ml.

### Pediatric Vaccines

Suitable immunogenic compositions include common childhood vaccines e.g. comprising one or more of:
an antigen from Neisseria meningitidis, such as a saccharide from one or more of serogroups A, C, W135 &/or Y (typically conjuagted)
an antigen from Streptococcus pneumoniae, such as a saccharide (typically conjugated)
an antigen from hepatitis B virus, such as the surface antigen HBsAg.
an antigen from Bordetella pertussis, such as pertussis holotoxin (PT) and filamentous haemagglutinin (FHA) from B.pertussis, optionally also in combination with pertactin and/or agglutinogens 2 and 3.
a diphtheria antigen, such as a diphtheria toxoid.
a tetanus antigen, such as a tetanus toxoid.
a saccharide antigen from Haemophilus influenzae B (Hib), typically conjugated.
inactivated poliovirus antigens, typically trivalent from polioviruses 1, 2 and 3
an influenza virus antigen, including whole inactivated and live attenuated influenza virus vaccines.

Other common childhood vaccines that may be used as the immunogenic composition of the invention include an MMR vaccine, a rotavirus vaccine, a varicella vaccine, a hepatitis A virus vaccine, etc.

Administering an immunomodulatory compound separately but simultaneously with, or prior or subsequent to, the administration of the childhood vaccine enhances the protective immunity elicited against the vaccine antigen(s). In some instances, the immune response may be enhanced in such a way that the number of boosters typically needed to yield a protective immune response is reduced.

For example, children typically receive three shots to raise a protective immune response against diphtheria, tetanus, pertussis, poliomyelitis, Haemophilus influenzae type B, Hepatitis B and pneumococcal capsular antigens. The three shots are usually administered during the first 12 months of a child's life. In one embodiment of the invention, the number of shots can be reduced from three to two, both of which can be administered during the first six-months of a child's life, if an immunomodulatory compound of the invention is administered separately but simultaneously with, or prior or subsequent to, the administration of the two shots. This is particularly advantageous because it reduces the overall cost of providing effective childhood immunisation by reducing the number of doses needed and the number of doctor office visits required.

Immunogenic composition that may be used in practising the invention could be any of the products sold as PENTACEL™, PEDIACEL™, HEXAVAC™, PEDIARIX™, INFANRIX PENTA™ INFANRIX HEXA™, QUINVAXEM™, EASYFIVE™, QUINTANRIX™, TRITANRIX™, TRITANRIX-HEPB™, ENGERIX B™ etc.

The immunogenic composition could also be any of the products sold as PREVNAR™ PREVNAR13™, SYNFLORIX™, etc.

The immunogenic composition could further be any of the products sold as MENJUGATE™, MENINGITEC™, NEISVAC-C™, MENACTRA™, MENVEO™, MENITORIX™, NIMENRIX™, MENHIBRIX™, etc.

The immunogenic composition could be any of the products sold as ROTARIX™, ROTATEQ™, GARDASIL™, CERVARIX™ etc.

### Vaccines for Adolescents

Suitable immunogenic compositions include common vaccines administered to adolescents and young adults.

The immunogenic composition could be any of the products sold as GARDASIL™, CERVARIX™ etc.

In particular, immunogenic compositions that are used to boost a pre-existing protective immunity established by a childhood vaccine may benefit from being administered in conjunction with an immunomodulatory compound of the invention.

The immunogenic composition could be any of the products sold as BOOSTRIX™, ADACEL™ etc.

### Influenza Vaccines

The immunomodulatory compound of the invention is of particular use in enhancing protective immunity in response to an influenza vaccine. Administration of the immunomodulatory compound and the influenza vaccine is especially useful for protecting the elderly and small children from seasonal or pandemic flu outbreaks. The immune system of elderly subject and small children is typically less able to mount and efficient immune response against a single influenza vaccination. To elicit a more effective immune response, one or more adjuvants are usually added to the immunogen to boost the response. However, the presence of one or more conventional adjuvant(s) may deter some people from getting vaccinated. While the use of adjuvants may not be completely avoided, the use of the immunomodulatory compounds in conjunction with flu vaccination may result in a more effective vaccination regimen that additionally results in fewer side effects (e.g. because the adjuvant dose and/or antigen dose can be reduced without affecting the efficacy of the vaccine).

Seasonal influenza vaccine that may could be any of the products sold as AGRIPPAL™ BEGRIVAC™ FLUAD™ OPTAFLU™, FLUMIST™, FLUVIRIN™ INFLUVAC™ FLUZONE™, FLUARIX™, etc.

The same rationale as for seasonal vaccines also applies for pandemic influenza vaccines. The pandemic influenza vaccine that may be used in practising the invention could be any of the products sold as DARONRIX™, FOCETRIA™, FOCLIVIA™, PANDEMRIX™, AREPANRIX™ CELVAPAN™, etc.

Pre-pandemic influenza vaccines are typically administered to subjects who have an increased risk of being exposed to a potentially pandemic influenza strain. This includes healthcare professionals, airline staff, and personnel in the food processing and farming sector. Administering the immunomodulatory compound separately but simultaneously with, or prior or subsequent to, the administration of a pre-pandemic influenza vaccine enhances the protective immunity elicited against the pre-pandemic vaccine and may result in better protection against exposure to a pandemic influenza strain. The pre-pandemic influenza vaccine could be any of the products sold as AFLUNOV™, PREPANDRIX™, etc.

### Cancer Vaccines

To prevent tumour recurrence, an effective cancer vaccine needs to trigger the formation of memory T cells against the cancer antigens found in the vaccine. The immunomodulatory compounds of the invention are believed to enhance the protective immunity partially by positively influencing the formation of memory T cells against antigens of an immunogenic composition. Thus, in one embodiment, administering an immunomnodulatory compound to a patient separately but simultaneously with, or prior or subsequent to, the administration of a cancer vaccine will improve the formation of cancer-specific memory T cells and decrease the recurrence of the cancer after it has been eliminated.

In another embodiment, administering the immunomodulatory compound of the invention with a cancer vaccine will result in a more effective anti-cancer immune response than the one observed in absence of the immunomodulatory compound, e.g. as measured by a more rapid or greater reduction in tumour size.

Cancer vaccines that might benefit from administration of the immunomodulatory compound include products currently being developed or sold as NEUVENGE™, PROVENGE™, ONCOPHAGE™ STIMUVAX™, NEUVAX™, CIMAVAX-EGF™, etc.

In one aspect of the invention, cancer vaccines are excluded as the immunogenic compositions in accordance with the invention.

### Drug-Carrier Conjugate Vaccines

The immunomodulatory compound of the invention may be of particular use in enhancing the immune response to drug-carrier conjugate vaccines that are designed to aid human subjects who are battling a drug addiction. The drug component is typically a hapten such as nicotine, cocaine, methamphetamine etc. Since haptens are generally not effective immunogens, they need to be conjugated to an immunogenic carrier protein that triggers an immune response and leads to the formation of anti-hapten antibodies. However, in order to prevent a small molecule drug to reach its target, a large and highly specific antibody response to the drug hapten is needed. Achieving such an effective immune response has been difficult with drug carrier vaccines administered alone or with an adjuvant.

By administering the immunomodulatory compound of the present invention simultaneously with, or prior or subsequent, to the administration of a drug-carrier conjugate vaccine, the immune response can be enhanced to achieve a high level of protection against the drug hapten in the majority of subjects. Drug-hapten carrier conjugates include products currently being developed as NICVAX™ TA-NIC, TA-CD, NIC002, etc.

Staphylococcus aureus Vaccine

Staphylococcus aureus is a Gram-positive spherical bacterium and is the leading cause of bloodstream, lower respiratory tract, skin and soft tissue infections. It causes a range of illnesses from minor skin infections to life-threatening diseases, and, in the US, annual mortality associated with S.aureus exceeds that of any other infectious disease, including HIV/AIDS.

All attempts to develop a protective vaccine against S.aureus have failed so far. The inventors surprisingly found that oral administration of NAM subsequent to administration of an S. aureus vaccine can enhance the protective immunity of mice against an otherwise lethal dose of S. aureus. Thus, in one particular aspect, the invention relates to a method for enhancing the immune response to an S. aureus vaccine. More specifically, the invention relates to a method for immunising a subject comprising administering to a subject (i) an immunogenic composition comprising an S. aureus antigen, and (ii) an immunomodulatory compound, wherein the immunogenic composition and the immunomodulatory compound are administered to the subject within 24 hours of each other. Preferably, the immunogenic composition comprises one or more of an EsxA antigen, an EsxB antigen, a Sta006 antigen, a Sta011 antigen and a Hla antigen.

The EsxA antigen and the EsxB antigen are preferably linked to form a hybrid polypeptide. Hence, in one embodiment, the invention relates to a method for enhancing the immune response to an S. aureus vaccine comprising administering to a subject (i) an immunogenic composition comprising a fusion protein containing an EsxA antigen and an EsxB antigen and a pharmaceutically acceptable carrier, and (ii) an immunomodulatory compound, wherein the immunogenic composition and the immunomodulatory compound are administered to the subject within 24 hours of each other.

For example, the immunogenic composition may comprise:
a first polypeptide comprising SEQ ID NO: 9, or a modified amino acid sequence which differs from SEQ ID NO: 9 by up to 5 single amino changes provided that the modified sequence can elicit antibodies which bind to a polypeptide consisting of SEQ ID NO: 9;
a second polypeptide comprising SEQ ID NO: 19, or a modified amino acid sequence which differs from SEQ ID NO: 19 by up to 5 single amino changes provided that the modified sequence can elicit antibodies which bind to a polypeptide consisting of SEQ ID NO: 19;
a third polypeptide comprising SEQ ID NO: 6, or a modified amino acid sequence which differs from SEQ ID NO: 6 by up to 5 single amino changes provided that the modified sequence can elicit antibodies which bind to a polypeptide consisting of SEQ ID NO: 6; and
a fourth polypeptide comprising SEQ ID NO: 12, or a modified amino acid sequence which differs from SEQ ID NO: 12 by up to 5 single amino changes provided that the modified sequence can elicit antibodies which bind to a polypeptide consisting of SEQ ID NO: 12.

The immunogenic composition comprising the S. aureus antigens may further comprise an adjuvant. Suitable adjuvants include aluminium salt adjuvants (e.g. aluminium hydroxide), oil-in-water emulsion adjuvants (e.g. MF59), or TLR agonists (such as the arginine salt of the compound with formula K2 above). In some instances, the immunogenic composition may be in lyophilized form. Preferably, the immunogenic composition comprising the S. aureus antigens is in aqueous form.

The invention further relates to an immunogenic composition comprising (i) an S. aureus antigen and (ii) an immunomodulatory compound for combined use in a method of immunising a human subject, wherein the immunogenic composition and the immunomodulatory compound are administered to the subject within 24 hours of each other.

The invention also relates to a combination of (i) immunogenic composition comprising an S. aureus antigen and (ii) an immunomodulatory compound for simultaneous, separate or sequential administration, wherein components (i) and (ii) are administered within 24 hours of each other.

In a further embodiment, the invention relates to a kit comprising (i) an immunogenic composition comprising an S. aureus antigen and (ii) an immunomodulatory compound. In another embodiment, the invention relates to a package comprising (i) an immunogenic composition comprising an S. aureus antigen and (ii) an information leaflet containing written instructions that an immunomodulatory compound may be administered to a subject within 24 hours of their receiving the immunogenic composition.

The use of nicotinamide and derivatives thereof and benzamide and derivatives thereof as immunomodulatory compounds are particularly preferred in practising the invention. However, an immunogenic composition comprising S. aureus antigens may be administered in conjunction with other immunomodulatory compounds to achieve the same effect, i.e. to enhance the protective immunity against the S. aureus antigens included in the vaccine. For example, thalidomide analogues such as lenalidomide and pomalidomide or tryptophan-derivatives such as SCV-07 (see references 1-3) may be used in practising the invention.

### EsxA

The ‘EsxA’ antigen is annotated as ‘protein’. In the NCTC 8325 strain EsxA is SAOUHSC_{—}00257 and has amino acid sequence SEQ ID NO: 1 (GI:88194063).

EsxA antigens of the invention can elicit an antibody (e.g. when administered to a human) that recognises SEQ ID NO: 1 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1; and/or (b) comprising a fragment of at least ‘n’ consecutive amino acids of SEQ ID NO: 1, wherein ‘n’ is 7 or more (e.g. 8, 10, 12, 14, 16, 18, 20, 25, 30, 35, 40, 50, 60, 70, 80, 90 or more). These EsxA proteins include variants of SEQ ID NO: 1. Preferred fragments of (b) comprise an epitope from SEQ ID NO: 10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 1 while retaining at least one epitope of SEQ ID NO: 1. Other fragments omit one or more protein domains.

### EsxB

The ‘EsxB’ antigen is annotated as ‘EsxB’. In the NCTC 8325 strain EsxB is SAOUHSC_{—}00265 and has amino acid sequence SEQ ID NO: 2 (GI:88194070).

EsxB antigens of the invention can elicit an antibody (e.g. when administered to a human) that recognises SEQ ID NO: 2 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2; and/or (b) comprising a fragment of at least ‘n’ consecutive amino acids of SEQ ID NO: 2, wherein ‘n’ is 7 or more (e.g. 8, 10, 12, 14, 16, 18, 20, 25, 30, 35, 40, 50, 60, 70, 80, 90, 100 or more). These EsxB proteins include variants of SEQ ID NO: 2. Preferred fragments of (b) comprise an epitope from SEQ ID NO: 2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 2 while retaining at least one epitope of SEQ ID NO: 2. Other fragments omit one or more protein domains.

### EsxAB

Where a composition includes both EsxA and EsxB antigens, these may be present as a single polypeptide (i.e. as a fusion polypeptide). Thus a single polypeptide can elicit antibodies (e.g. when administered to a human) that recognise both SEQ ID NO: 1 and SEQ ID NO: 2. The single polypeptide can include: (i) a first polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1 and/or comprising a fragment of at least ‘n’ consecutive amino acids of SEQ ID NO: 1, as defined above for EsxA; and (ii) a second polypeptide sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2 and/or comprising a fragment of at least ‘n’ consecutive amino acids of SEQ ID NO: 2, as defined above for EsxB. The first and second polypeptide sequences can be in either order, N- to C-terminus. SEQ ID NOs: 3 (‘EsxAB’) and 4 (‘EsxBA’) are examples of such polypeptides, both having hexapeptide linkers ASGGGS (SEQ ID NO: 5). Another ‘EsxAB’ hybrid comprises SEQ ID NO: 6, which may be provided with an N-terminal methionine (e.g. SEQ ID NO: 7).

Thus a useful polypeptide comprises an amino acid sequence (a) having 80% or more identity (e.g. 80%, 85%, 90%, 91%, 92%, 93%, 94%, 95%, 96%, 97%, 98%, 99%, 99.5% or more) to SEQ ID NO: 6; and/or (b) comprising both a fragment of at least ‘n’ consecutive amino acids from amino acids 1-96 of SEQ ID NO: 6 and a fragment of at least ‘n’ consecutive amino acids from amino acids 103-205 of SEQ ID NO: 6, wherein ‘n’ is 7 or more (e.g. 8, 10, 12, 14, 16, 18, 20, 25, 30, 35, 40, 50, 60, 70, 80, 90, 100, 150, 200, 250 or more). These polypeptides (e.g. SEQ ID NO: 7) can elicit antibodies (e.g. when administered to a human) which recognise both the wild-type staphylococcal protein comprising SEQ ID NO: 1 and the wild-type staphylococcal protein comprising SEQ ID NO: 2. Thus the immune response will recognise both of antigens EsxA and EsxB. Preferred fragments of (b) provide an epitope from SEQ ID NO: 1 and an epitope from SEQ ID NO: 2.

### Sta006

The ‘Sta006’ antigen is annotated as ‘ferrichrome-binding protein’, and has also been referred to as ThuD2′ in the literature [72]. In the NCTC 8325 strain Sta006 is SAOUHSC_{—}02554 and has amino acid sequence SEQ ID NO: 8 (GI:88196199). In the Newman strain it is nwmn_{—}2185 (GI:151222397). Sta006 used with the invention can elicit an antibody (e.g. when administered to a human) that recognises SEQ ID NO: 8 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) comprising a fragment of at least ‘n’ consecutive amino acids of SEQ ID NO: 8, wherein ‘n’ is 7 or more (e.g. 8, 10, 12, 14, 16, 18, 20, 25, 30, 35, 40, 50, 60, 70, 80, 90, 100, 150, 200, 250 or more). These Sta006 proteins include variants of SEQ ID NO: 8. Preferred fragments of (b) comprise an epitope from SEQ ID NO: 8. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 8 while retaining at least one epitope of SEQ ID NO: 8. The first 17 N-terminal amino acids of SEQ ID NO: 8 can usefully be omitted (to provide SEQ ID NO: 9). Other fragments omit one or more protein domains. Mutant forms of Sta006 are reported in reference 73. A Sta006 antigen may be lipidated e.g. with an acylated N-terminus cysteine. One useful Sta006 sequence is SEQ ID NO: 10, which has a Met-Ala-Ser-sequence at the N-terminus. Sta006 can exist as a monomer or an oligomer (e.g. dimer), with Ca′ ions favouring oligomerization. The invention can use monomers and/or oligomers of Sta006. Sta006 can be a homodimer or heterodimer with Sta011.

### Sta011

The ‘Sta011’ antigen is annotated as ‘lipoprotein’. In the NCTC 8325 strain Sta011 is SAOUHSC_{—}00052 and has amino acid sequence SEQ ID NO: 11 (GI:88193872).

Sta011 antigens used with the invention can elicit an antibody (e.g. when administered to a human) that recognises SEQ ID NO: 11 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). These Sta011 proteins include variants of SEQ ID NO: 11. Preferred fragments of (b) comprise an epitope from SEQ ID NO: 11. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 11 while retaining at least one epitope of SEQ ID NO: 11. The first 23 N-terminal amino acids of SEQ ID NO: 11 can usefully be omitted (to provide SEQ ID NO: 12). Other fragments omit one or more protein domains. A Sta011 antigen may be lipidated e.g. with an acylated N-terminus cysteine. One useful Sta011 sequence is SEQ ID NO: 13, which has an N-terminal methionine. Variant forms of SEQ ID NO: 11 which may be used as or for preparing Sta011 antigens include, but are not limited to, SEQ ID NOs: 14, 15 and 16 with various Ile/Val/Leu substitutions. Sta011 can exist as a monomer or an oligomer, with Ca′ ions favouring oligomerisation. The invention can use monomers and/or oligomers of Sta011.

### Hla

The ‘Hla’ antigen is the ‘alpha-hemolysin precursor’ also known as ‘alpha toxin’ or simply ‘hemolysin’. In the NCTC 8325 strain Hla is SAOUHSC 01121 and has amino acid sequence SEQ ID NO: 17 (GI:88194865). Hla is an important virulence determinant produced by most strains of S.aureus, having pore-forming and haemolytic activity. Anti-Hla antibodies can neutralise the detrimental effects of the toxin in animal models, and Hla is particularly useful for protecting against pneumonia.

Hla antigens used with the invention can elicit an antibody (e.g. when administered to a human) that recognises SEQ ID NO: 17 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 17; and/or (b) comprising a fragment of at least ‘n’ consecutive amino acids of SEQ ID NO: 17, wherein ‘n’ is 7 or more (e.g. 8, 10, 12, 14, 16, 18, 20, 25, 30, 35, 40, 50, 60, 70, 80, 90, 100, 150, 200, 250 or more). These Hla proteins include variants of SEQ ID NO: 17. Preferred fragments of (b) comprise an epitope from SEQ ID NO: 17. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 17 while retaining at least one epitope of SEQ ID NO: 17. The first 26 N-terminal amino acids of SEQ ID NO: 17 can usefully be omitted (e.g. to give SEQ ID NO: 18). Truncation at the C-terminus can also be used e.g. leaving only 50 amino acids (residues 27-76 of SEQ ID NO: 17) [74]. Other fragments omit one or more protein domains.

Hla's toxicity can be avoided in compositions of the invention by chemical inactivation (e.g. using formaldehyde, glutaraldehyde or other cross-linking reagents). Instead, however, it is preferred to use mutant forms of Hla which remove its toxic activity while retaining its immunogenicity. Such detoxified mutants are already known in the art. One useful Hla antigen has a mutation at residue 61 of SEQ ID NO: 17, which is residue 35 of the mature antigen (i.e. after omitting the first 26 N-terminal amino acids=residue 35 of SEQ ID NO: 18). Thus residue 61 may not be histidine, and may instead be e.g. Ile, Val or preferably Leu. A His-Arg mutation at this position can also be used. For example, SEQ ID NO: 19 is the mature mutant Hla-H35L sequence (i.e. SEQ ID NO: 18 with a H35L mutation) and a useful Hla antigen comprises SEQ ID NO: 19. Another useful mutation replaces a long loop with a short sequence e.g. to replace the 39mer at residues 136-174 of SEQ ID NO: 17 with a tetramer such as PSGS (SEQ ID NO: 20), as in SEQ ID NO: 21 (which also includes the H35L mutation) and SEQ ID NO: 22 (which does not include the H35L mutation). Another useful mutation replaces residue Y101 e.g. with a leucine (SEQ ID NO: 23). Another useful mutation replaces residue D152 e.g. with a leucine (SEQ ID NO: 24). Another useful mutant replaces residues H35 and Y101 e.g. with a leucine (SEQ ID NO: 25). Another useful mutant replaces residues H35 and D152 e.g. with a leucine (SEQ ID NO: 26).

Further useful Hla antigens are disclosed in references 75 and 76.

SEQ ID NOs: 27, 28 & 29 are three useful fragments of SEQ ID NO: 17 (‘Hla₂₇₋₇₆’, ‘Hla₂₇₋₈₉’ and ‘Hla₂₇₋₇₉’, respectively). SEQ ID NOs: 30, 31 and 32 are the corresponding fragments from SEQ ID NO: 19.

One useful Hla sequence is SEQ ID NO: 33, which was used in the examples. It has an N-terminal Met, then an Ala-Ser dipeptide from the expression vector, then SEQ ID NO: 19 (from NCTC8325 strain). It is encoded by SEQ ID NO: 34.

### Methods of Treatment

The invention relates to a method for immunising a human subject, comprising administering to a human subject (i) an immunogenic composition and (ii) an immunomodulatory compound. In some instances, more than one immunogenic composition is administered at the same time. For example, certain immunogenic compositions may be sold or packaged separately or are included as a separate component of a kit. They may be administered separately but at the same time or substantially at the same time, e.g. by being injected proximal to the injection site or in opposite limbs during a single doctor's visit. The immunogenic composition and immunomodulatory compound may be administered separately at the same time. Alternatively, the immunomodulatory compound is administered prior to or subsequent to the administration of the immunogenic compound. For example, the immunogenic composition and the immunomodulatory compound are administered within 24 hours of each other. They may be administered within 12 hours of each other e.g. within 6 hours of each other, within 3 hours of each other, within 2 hours of each other, within 1 hour of each other, within 30 minutes of each other, within 20 minutes of each other, within 10 minutes of each other, or within 5 minutes of each other. Preferably, the immunomodulatory compound is administered after the administration of the immunogenic composition. Most preferably, administration of the immunomodulatory compound will occur for the first time at least 24 hours after the administration of the immunogenic composition.

The inventors believe that the immunomodulatory compounds of the present invention modulate the expression of at least two classes of genes in cells of the innate immune system. The immunomodulatory compounds influence the expression of proinflammatory cytokines such as IL-1β, IL-6, IL-8 and TNF-α and positively affect the expression of genes encoding antimicrobial peptides. It is the inventor's belief that the immunomodulatory compounds described herein are thus able to condition the immune response to an immunogen in such a way that it yields greater protective immunity. The initial cytokine response by antigen-presenting cells encountering the immunogen is believed to shape the subsequent events that occur during an adaptive immune response by activating a specific subset of T cells. Without being bound by any particular theory, the inventors believe that immunomodulatory compounds may exert a positive effect on the immune response against pathogens by inducing cytokines that are capable of facilitating the recruitment and/or activation of phagocytes.

Therefore, administering the immunomodulatory compound before, after or simultaneously with the administration of an immunogenic composition will result in similar beneficial effects as long as a pharmacological effective amount of the compound is present while the immunogen is processed and/or presented to T-cells by antigen presenting cells. Administering the immunomodulatory compound for the first time at least 24 hours after the administration of the immunogenic composition appears to be most effective in enhancing the protective immune response and therefore is a preferred embodiment of the invention.

In a typical embodiment of the invention, the immunomodulatory compound will be administered to the subject for the first time at least 24 hours after administration of the immunogenic composition e.g. within 5 days, 7 days, 10 days, 14 days, 21 days, 1 month, 3 months, 6 months, 1 year, 2 years, 10 years, after administration of the immunogenic composition. For example, the immunomodulatory compound may be administered within 5-21 days post vaccination, preferable within 7-10 days post vaccination. In some instances, the time interval may be shorter and the immunomodulatory compound is administered for the first time within 24 to 48 hours, 24 to 72 hours, or 24 to 96 hours from administration of the immunogenic composition.

The invention may involve more than one administration of the immunomodulatory compound e.g. the invention may involve 1, 2, 3, 4 or more administrations of the immunomodulatory compound. Where more than one administration of the immunomodulatory compound is given then the above timing (i.e. within 10 years of each other, down to within 5 minutes of each other) refers to the shortest period between administration of the immunogenic composition and administration of the immunomodulatory compound. For example, a subject may receive the immunogenic composition once and the immunomodulatory at least twice within a 24 hour period, preferably a 48 hour period, and more preferably a 96 hour period.

Where the invention does involve more than one administration of the immunomodulatory compound, these (i) can all be before administration of the immunogenic composition, (ii) can all be after administration of the immunogenic composition, (iii) can span administration of the immunogenic composition, with at least one before and at least one after, or (iv) can involve at least one administration before and/or after, together with one administration at the same time as the immunogenic composition.

In some instances, the immunomodulatory compound may be administered to the subject over a period of time after the administration of at least one immunogenic composition. For example, the immunomodulatory compound may be administered daily for at least 2 days, 3 days, 4 days or 5 days after administration of the immunogenic composition beginning from the day of administration.

Alternatively, the immunomodulatory compound may be administered to the subject over a period of time before the administration of at least one immunogenic composition. For example, the immunomodulatory compound may be administered daily for at least 2 days, 3 days, 4 days or 5 days before administration of the immunogenic composition up to or including the day on which the immunogenic composition is administered.

In another typical embodiment, the invention involves: (i) administration of an immunomodulatory compound; then (ii) within 24 hours of step (i), administration of an immunogenic composition; then (iii) one or two further doses of the immunomodulatory compound, and possibly a third, after step (ii).

In certain aspects of the invention, the subject has received an immunogenic composition at least one week, two weeks, three weeks or four weeks prior to a second exposure to an antigen that formed part of the immunogenic composition. In some embodiments, the subject also received an immunomodulatory compound during a 24-hour time period before or after administration of the immunogenic composition. The second exposure is typically in the form of a live pathogen belonging to the same or related species from which the antigen found in the immunogenic composition was derived. For instance, the second exposure may occur in a hospital setting in the form of a pathogen that causes nosocomial infections such as S. aureus, C. albicans, S. pyogenes, etc.

For example, a subject may be scheduled to undergo a planned operation at a hospital and has received an S. aureus vaccine at least one week, two weeks, three weeks or four weeks prior to being admitted to a hospital. During the 24-hour time period before or after the (suspected) second exposure (e.g. 24 hours before being admitted to the hospital), the subject will then be administered one or more dose of an immunomodulatory compound to enhance the protective effect of the vaccine upon a second exposure to an S. aureus antigen in the form of live bacteria in the hospital. Alternatively, administration of the immunomodulatory compound may begin at least 24 hours before the (suspected) second exposure, and may be continued afterwards in certain intervals, e.g. every 12 hours, every 24 hours, every 48 hours.

Similarly, a subject may be vaccinated with a prepandemic or pandemic influenza vaccine at least one week, two weeks, three weeks, four weeks, one month, three months, 6 months, 1 year, 2 years prior to the expected exposure to a pandemic influenza strain. The subject will then be administered one or more dose of an immunomodulatory compound before or during the period in which exposure to the pandemic influenza strain is suspected. Administration of the immunomodulatory compound may continue for the entire time period in which exposure may be possible. Doses may be administered every 12 hours, every 24 hours, every 48 hours etc.

The same rationale may apply to a subject who is scheduled to visit a region of the world in which certain infectious diseases (e.g. yellow fever, hepatitis A and B, typhoid, rabies etc.) are prevalent and which the subject would normally not encounter in his home country. The subject will have received vaccination for any such disease at least one week, two weeks, three weeks or four weeks, one month, three months, 6 months, 1 year, 2 years, 10 years prior to his or her departure from his or her home country. The subject will then be administered one or more dose of an immunomodulatory compound during the 24-hour period before his or her departure or after his or her arrival at the destination to enhance the protective effect of the vaccine(s) prior to (suspected) exposure to a pathogen prevalent at his or her travel destination. Administration of the immunomodulatory compound may be continued after arrival at the travel destination. For example, additional doses may be administered in intervals of 12 hours, 24 hours, 48 hours etc.

Administration of the immunomodulatory compound and the immunogenic composition can be performed by the same person or by different persons. The immunogenic composition will generally be administered by a healthcare professional (e.g. physician, nurse) whereas the immunomodulatory compound can be self-administered.

Generally, the immunogenic composition will be administered by injection (e.g. intramuscular injection), whereas the immunomodulatory compound may be administered orally (e.g. by tablet or capsule, or by liquid oral suspension) or by injection depending on the nature of the compounds. For example nicotinamide and nicotinamide derivatives are preferably administered orally, whereas TLR agonists are typically administered by injection.

The subject

The invention is useful for enhancing the protective immunity of a subject in response to an immunogenic composition. The subject can be any animal capable of forming B- or T-cell-mediated immune responses including humans, livestock and pets.

Preferably, the invention is used to enhance the protective immunity of a human subject in response to a vaccine containing one or more immunogen(s). It can be used with children and adults, and so the human subject may be less than 1 year old, 1-5 years old, 2-11 years old, 5-15 years old, 12-21 years old, 15-55 years old, or at least 55 years old. Enhancing the protective immunity elicited by an immunogen is of particular interest in infants and toddlers, and so the subject is preferably less than 1 year old (e.g. between 0-6 months old) or is between 1-5 years old. The capacity to mount a protective immune response diminishes with age. Hence the invention may also be useful in human subjects over the age of 45, preferable over the age of 50, more preferably over the age of 60.

The invention may also be useful in human subjects who are immunocompromised due to illness or pharmacological intervention. This includes transplant and cancer patients who have received drugs that down-modulate the immune response (e.g., cyclosporin) or affect the health or survival of immune cells (e.g., cytostatic agents).

The human subject can be in any ethnic or racial group.

The human subject may already have received at least one previous vaccine. Thus the subject's immune system may have been previously exposed to vaccine antigens e.g. to diphtheria toxoid (Dt), tetanus toxoid (Tt). Thus the subject may previously have raised an anti-Dt antibody response (typically to give an anti-Dt titer >0.01 IU/ml) and will possess memory B and/or T lymphocytes specific for Dt. Similarly, the subject may previously have raised an anti-Tt antibody response (typically to give an anti-Tt titer >0.01 IU/ml) and will possess memory B and/or T lymphocytes specific for Tt. Thus the subject may be distinct from subjects in general, as they are members of a subset of the general population whose immune systems have already mounted an immune response to e.g. Dt and/or Tt. As well as having been previously exposed to Dt or Tt, the subject may previously have received other antigens e.g. pertussis antigen(s), Haemophilus influenzae type B capsular saccharide, hepatitis B virus surface antigen (HBsAg), inactivated poliovirus vaccine, Streptococcus pneumoniae capsular saccharides, influenza virus vaccine, BCG, measles virus, mumps virus, rubella virus, varicella virus, N.meningitidis capsular saccharide(s), S. aureus (combination) vaccine etc.

In one aspect of the invention, the subject has previously received a vaccine against a pathogen causing one or more of the following diseases: yellow fever, hepatitis, typhoid, rabies, a staphylococcal infection, malaria, meningitis, and encephalitis. In a specific embodiment, the subject has received as vaccine against a traveller's disease (including hepatitis A and B, yellow fever, typhoid, rabies, malaria, and Japanese encephalitis). In another specific embodiment, the subject has received a vaccine against a nosocomial infection (such as an infection with S. aureus, Candida albicans, Streptococcus pyogenes, etc.). In yet another specific embodiment, the subject has received a prepandemic or pandemic influenza vaccine.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references 77-83, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

### DESCRIPTION OF THE DRAWINGS

FIG. 1: Chart showing survival of mice within 15-day period after infection with a lethal dose of S. aureus.

FIG. 2: Chart showing antibody response against HlaH35L at day 23 after vaccination (LLOQ=lower limit of quantitation).

FIG. 3: Chart showing antibody response against Sta006 at day 23 after vaccination (LLOQ=lower limit of quantitation).

FIG. 4: Chart showing antibody response against Sta011 at day 23 after vaccination (LLOQ=lower limit of quantitation).

FIG. 5: Chart showing antibody response against EsxAB at day 23 after vaccination (LLOQ=lower limit of quantitation).

### MODES FOR CARRYING OUT THE INVENTION

### Example 1

An S. aureus combination vaccine was prepared by mixing the purified recombinant S. aureus antigens HlaH35L, EsxAB, Sta006, Sta011. The proteins were adsorbed to aluminium hydroxide adjuvant (alum, 2 mg/ml) and the final formulation contained 50 μg protein/ml of each antigen. The pH and the osmolality of the formulations were within the optimal pH range (6.5-7) and osmolality (0.300 Osm/kg+/−0.020 Osm/kg).

TABLE 1
Antigens in S. aureus combination vaccine

| | | | SEQ |
|---|---|---|---|
| | | | ID |
| Antigen | Modification in S. aureus | Size/MW | NO. |
| EsxAB | Wild type EsxA and EsxB fused | 206 amino acids/ | 7 |
| | with a short spacer (ASGGGS) | 22.8 kDa | |
| Sta006 | Wild type Sta006 | 288 amino acids/ | 10 |
| | | 32 kDa | |
| Sta011 | Wild type Sta011 | 234 amino acids/ | 13 |
| | | 27 kDa | |
| HlaH35L | Hla detoxified by one amino acid | 396 amino acids/ | 33 |
| | substation (His35Leu) | 33 kDa | |

### Example 2

Four groups of 47 to 48 CD1 mice were treated as follows: CD1 mice were immunized twice via intraperitoneal injection two weeks apart (on day 0 and day 14), and each mouse received 200 μl of (mock-)vaccine. Groups 1 (Alum-Nam) and 2 (Alum) were mock-injected with alum (saline plus 2 mg/ml aluminium-hydroxide), groups 3 (Combo-NAM) and 4 (Combo only) were immunised with the S. aureus combination vaccine of Example 1. Groups 1 (Alum-Nam) and 3 (Combo-NAM) received 250 mg/kg NAM orally one day before and one day after challenge with a lethal dose of live S. aureus.

On day 24 (i.e. 10 days after the second vaccination), the immunised animals of groups 1-4 were challenged by intraperitoneal injection of a bacterial suspension of S. aureus strain Newman (approximately 2 to 5×10⁸ CFU). Cultures of S. aureus were centrifuged, washed twice and diluted in PBS before challenge. Survival of the mice was monitored over a 15-day period post infection.

Survival rates were analysed by Mann-Whitney U-test. Mice were daily monitored and euthanized according to humane endpoints, in agreement with Novartis Animal Welfare Policies.

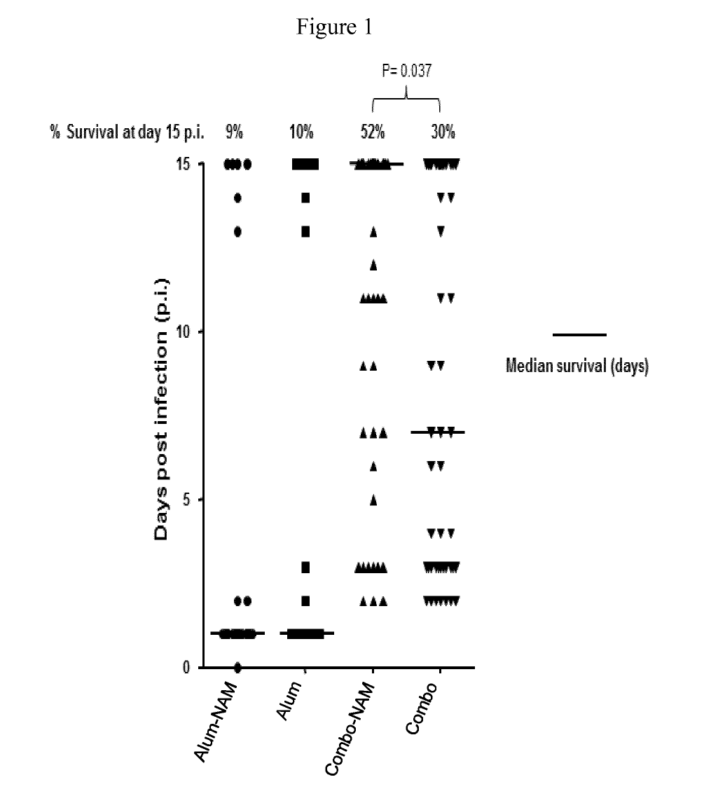

The results of the survival study are summarised in FIG. 1.

Administration of NAM to mice treated with alum alone was not effective in protecting mice against a lethal infection with S. aureus. Of the mice in groups 1 (Alum-Nam) and 2 (Alum), only 9% and 10%, respectively, survived until 15 days post infection (p.i.), at which point the study was terminated. However, when NAM was administered to mice immunized with an S. aureus combination vaccine, mortality could be significantly decreased in comparison to mice which had received only the vaccine. Of the 48 mice in group 3(Combo-NAM), 52% were still alive at day 15, whereas only 30% of the 47 mice in group 4 (Combo only) survived until day 15. The difference in survival between groups 3 (Combo-NAM) and 4 (Combo only) was statistically significant (P=0.037).

Thus oral administration of NAM was able to enhance the protective immunity in mice vaccinated with the S. aureus combination vaccine and prolong the survival mice during a subsequent challenge with a lethal dose of S. aureus.

### Example 3

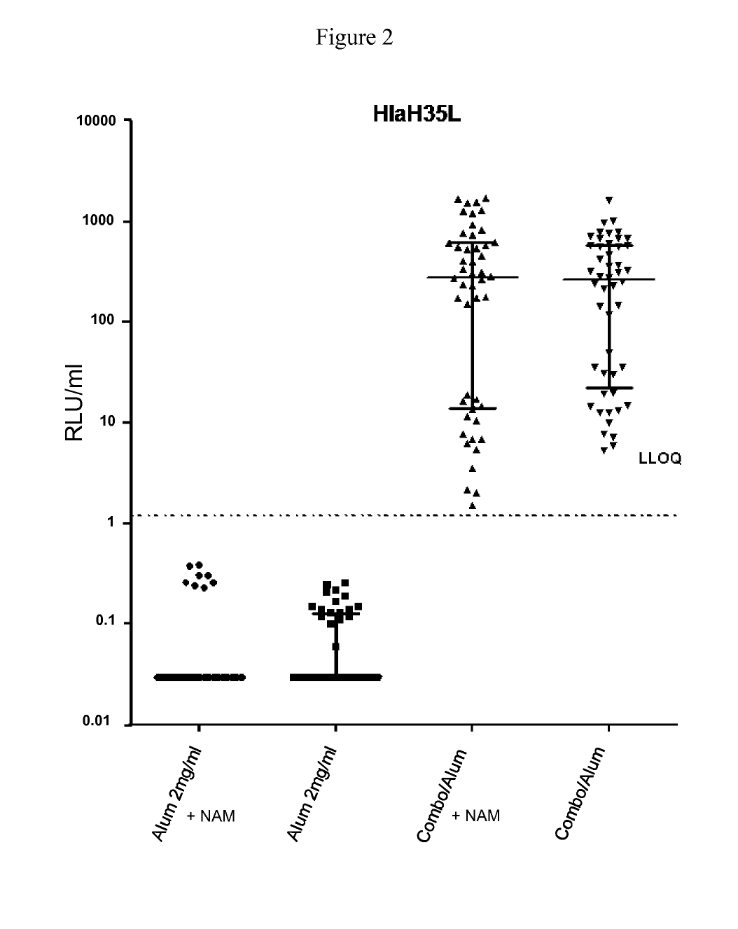

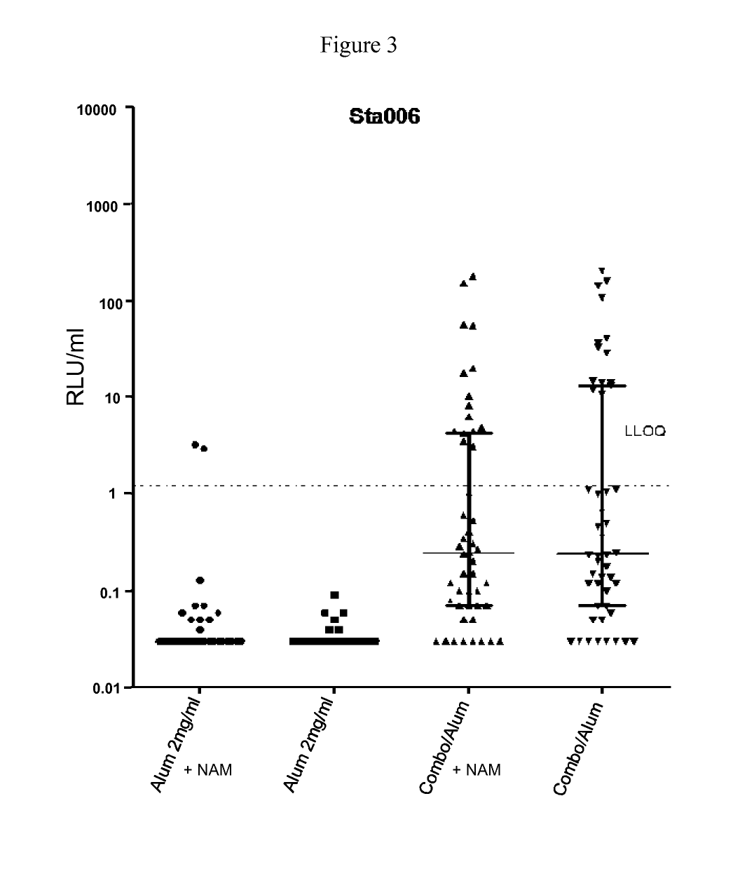

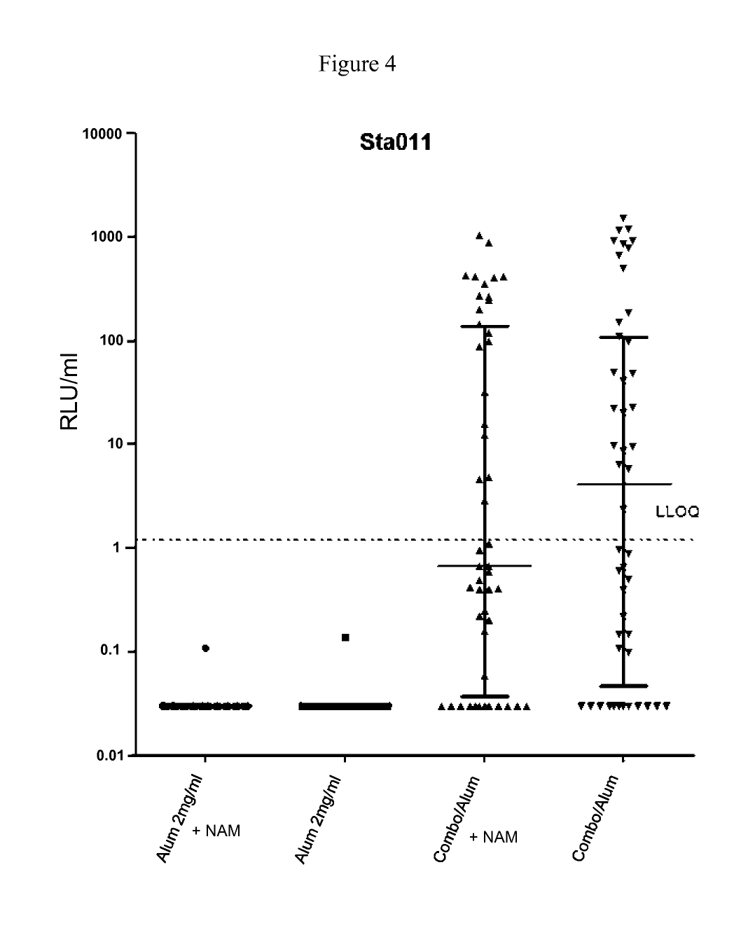

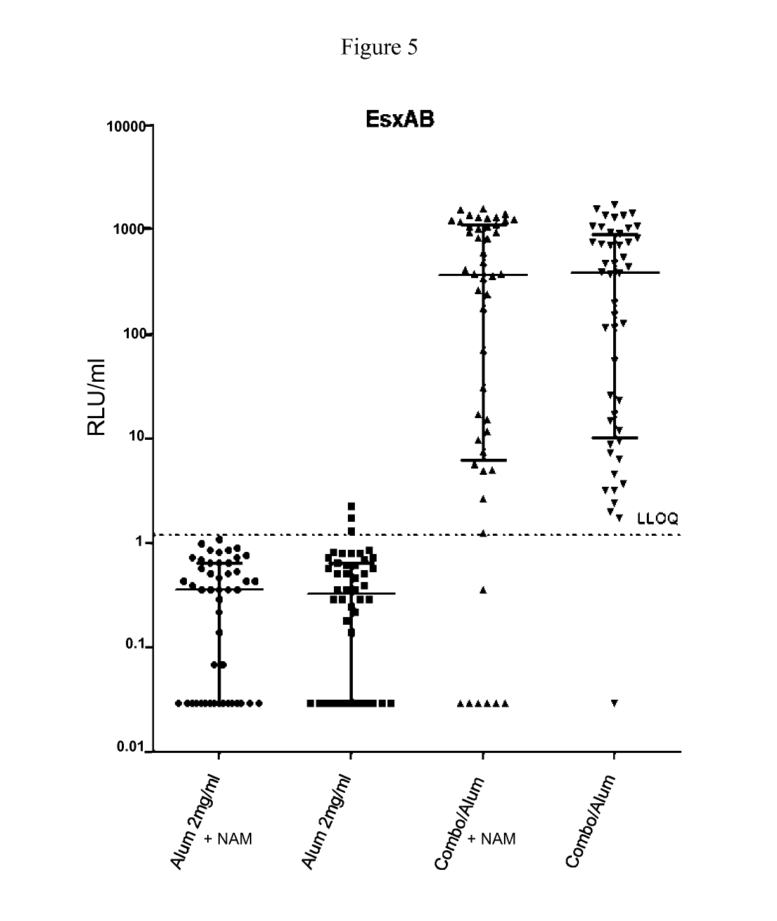

At day 23 after the first vaccination with the S. aureus combination vaccine (i.e. one day prior to the challenge with a lethal dose of S. aureus), the HlaH35L, EsxAB, Sta006 and Sta011 antibody titres for each group of mice described in Example 2 were measured by Luminex assay. The results for each of the four antigens present in the combination vaccine are summarised in FIGS. 2-5, respectively. In FIGS. 2-5:
Group 1 is Alum 2 mg/ml+NAM (first column) and corresponds to the group of mice which were mock-injected with alum (saline plus 2 mg/ml aluminium-hydroxide) and which received 250 mg/kg NAM orally one day before and one day after challenge with a lethal dose of live S. aureus.
Group 2 is Alum 2 mg/ml (second column) and corresponds to the group of mice which were mock-injected with alum (saline plus 2 mg/ml aluminium-hydroxide) but this group did not receive NAM.
Group 3 is Combo/Alum+NAM (third column) and corresponds to the group of mice which were immunised with the S. aureus combination vaccine of Example 1 and which received 250 mg/kg NAM orally one day before and one day after challenge with a lethal dose of live S. aureus.
Group 4 is Combo/Alum (fourth column) and corresponds to the group of mice which were immunised with the S. aureus combination vaccine of Example 1 but this group did not receive NAM.

No significant difference was observed between the HlaH35L, EsxAB, Sta006 and Sta011 antibody titres in the serum of mice in group 3 (vaccinated, NAM treatment) and the HlaH35L, EsxAB, Sta006 and Sta011 antibody titres in the serum of mice in group 4 (vaccinated, no NAM treatment).

These results demonstrate that the enhanced protective immunity observed in vaccinated mice treated with NAM was not due to the stimulation of the adaptive immune response.

It will be understood that the invention is described above by way of example only and modifications may be made whilst remaining within the scope and spirit of the invention.

### REFERENCES

[1] WO2007/028047
[2] Dredge et al. (2002) J Immunol. 168(10):4914-9
[3] US2009/0074815
[4] Murray (2003) Clin. Infect. Dis. 36(4):453-60
[5] Jordahl et al. (1961) Am. Rev. Respir. Dis. 83:899-900
[6] Pozzilli et al. (1997) Lancet 349(9064):1520-1
[7] WO2011/133692
[8] Kyme et al. (2012) J. Clin. Invest. 122(9):3316-29
[9] Niren (2006) Cutis 77 (1 Suppl):11-6
[10] Ungerstedt et al. (2003) Clin. Exp. Immunol. 131(1):48-52
[11] Pero et al. (1999) Mol. Cell. Biochem. 193 (1-2):119-25
[12] EP2070908
[13] US6380218B1
[14] US6649633B2
[15] Lindgren et al. (2001) Mol. Immunol. 38(4):267-77
[16] Liberg et al. (1999) Br. J. Cance. 1999 81(6):981-8
[17] W01997/32582
[18] Knip et al. (2000) Diabetologia 43(11):1337-45
[19] Redfern et al. (2011) Exp Eye Res 92(3):209-20
[20] Nava-Parada et al. (2007) Cancer Res 67(3):1326-34
[21] Zheng et al. (2008) Cancer Res 68(11):4045-9
[22] Taillardet et al. (2010) J Infect Dis 202(3):470-9
[23] Jensen et al. (2012) Infect Immun 80(5):1744-52
[24] Foster et al. (2007) Nature 447(7147):972-8
[25] Foster & Medzhitov (2009) Clin Immunol 130(1):7-15
[26] Rosenberg et al. (2010) J Immunol 184:136.20
[27] U.S. Pat. No. 4,666,886
[28] WO2009/118296
[29] WO2008/005555
[30] WO2009/111337
[31] WO2009/067081
[32] WO2007/040840
[33] WO2010/014913
[34] WO2011/119759
[35] WO2012/031140
[36] WO2007/034917
[37] WO2007/034173
[38] WO2008/114817
[39] US2009-0105212
[40] US2009-0118263
[41] US2009-0143400
[42] US2009-0192153
[43] WO2007/093901
[44] WO2009/019553
[45] US2009/0221631
[46] WO2008/004948
[47] WO2008/135791
[48] US2009/0099216
[49] US2009/0202484
[50] WO2008/101867
[51] WO2010/077613
[52] US2010/0143301
[53] Steinhagen et al. (2011) Vaccine 29:3341-55
[54] GB-A-2220211
[55] Myers et al. (1990) pages 145-156 of Cellular and Molecular Aspects of Endotoxin Reactions
[56] Ulrich (2000) Chapter 16 (pages 273-282) in: Vaccine Adjuvants: Preparation Methods and Research Protocols (Volume 42 of Methods in Molecular Medicine series). ISBN: 1-59259-083-7. Ed. O'Hagan
[57] Johnson et al. (1999) J Med Chem 42:4640-9
[58] Baldrick et al. (2002) Regulatory Toxicol Pharmacol 35:398-413
[59] Coler et al. (2011) PLoS ONE 6 (1):e16333.
[60] Johnson et al. (1999) Bioorg Med Chem Lett 9:2273-2278
[61] Evans et al. (2003) Expert Rev Vaccines 2:219-229
[62] Bazin et al. (2006) Tetrahedron Lett 47:2087-92
[63] Wong et al. (2003) J Clin Pharmacol 43(7):735-42
[64] US2005/0215517
[65] WO2003/011223
[66] WO2007/053455
[67] Garcon et al. (2007) Expert Rev Vaccines 6:723-39
[68] W01994/21292
[69] WO2011/027222
[70] Gennaro (2000) Remington: The Science and Practice of Pharmacy. 20th edition, ISBN: 0683306472
[71] Vaccine Design (1995) eds. Powell & Newman. ISBN: 030644867X. Plenum
[72] Sebulsky & Heinrichs (2001) J Bacteriol 183:4994-5000
[73] Sebulsky et al. (2003) J Biol Chem 278:49890-900
[74] Rable & Wardenburg (2009) Infect Immun 77:2712-8
[75] WO2007/145689
[76] WO2009/029831
[77] Methods In Enzymology (S. Colowick and N. Kaplan, eds., Academic Press, Inc.)
[78] Handbook of Experimental Immunology, Vols. I-IV (D. M. Weir and C. C. Blackwell, eds, 1986, Blackwell Scientific Publications)
[79] Sambrook et al. (2001) Molecular Cloning: A Laboratory Manual, 3rd edition (Cold Spring Harbor Laboratory Press)
[80] Handbook of Surface and Colloidal Chemistry (Birdi, K. S. ed., CRC Press, 1997)
[81] Ausubel et al. (eds) (2002) Short protocols in molecular biology, 5th edition (Current Protocols).
[82] Molecular Biology Techniques: An Intensive Laboratory Course, (Ream et al., eds., 1998, Academic Press)
[83] PCR (Introduction to Biotechniques Series), 2nd ed. (Newton & Graham eds., 1997, Springer Verlag)

1. A method for immunising a subject, comprising administering to a subject (i) at least one immunogenic composition and (ii) an immunomodulatory compound other than lenalidomide and pomalidomide, wherein the immunomodulatory compound is administered to the subject for the first time more than 24 hours after administration of the immunogenic composition.

2. The method of claim 1, wherein the immunomodulatory compound is an innate immunity stimulator.

3. The method of claim 2, wherein the innate immunity stimulator is a TLR agonist.

4. The method of claim 2, wherein the innate immunity stimulator is a compound of formula I: 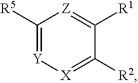
or a pharmaceutically acceptable salt thereof, wherein:
X is selected from N and CR3;
Y is selected from N and CR4;
Z is selected from N and CR6;
R1 is selected from C(O)NR7R8, NR7R8 and NR7C(O)R8;
each of R2, R3, R4, R5, R6, R7 and R8 is independently selected from hydrogen, hydroxyl, cyano, nitro, C1-6-alkyl, C2-6-alkenyl, C2-6-alkynyl, C1-6-alkoxy, C2-6-alkenyloxy, C2-6-alkynyloxy, halogen, C1-6-alkylcarbonyl, carboxy, C1-6-alkoxycarbonyl, amino, C1-6-alkylamino, di-C1-6-alkylamino, C1-6-alkylaminocarbonyl, di-C1-6-alkylaminocarbonyl, C1-6-alkylcarbonylamino, C1-6-alkylcarbonyl(C1-6-alkyl)amino, C1-6-alkylsulfonyl amino, C1-6-alkylsulfonyl(C1-6-alkyl)amino, C1-6-thioalkyl, C1-6-alkylsulfinyl, C1-6-alkylsulfonyl, aminosulfonyl, C1-6-alkylaminosulfonyl and di-C1-6-alkylaminosulfonyl, optionally wherein each of the aforementioned hydrocarbon groups is substituted by one or more halogen, hydroxyl, C1-6-alkoxy, amino, C1-6-alkylamino, and di-C1-6-alkylamino or cyano.

5. (canceled)

6. A combination of (i) an immunogenic composition comprising one or more S. aureus antigen(s) selected from the group consisting of HlaH35L, EsxAB, Sta006 and Sta011; and (ii) an immunomodulatory compound as defined in claim 29, for separate or sequential administration, wherein the immunomodulatory compound is administered for the first time more than 24 hours after administration of the immunogenic composition.

7. A kit comprising (i) an immunogenic composition comprising one or more S. aureus antigen(s) selected from the group consisting of HlaH35L, EsxAB, Sta006 and Sta011; and (ii) an immunomodulatory compound as defined in claim 29.

8. (canceled)

9. A method for immunising a subject, comprising administering to a subject (i) a first dose of an immunogenic composition as a prime, (ii) a second dose of the immunogenic composition as a boost, and (iii) an immunomodulatory compound, wherein administration of the first dose and the second dose are at least one month apart and administration of the immunomodulatory compound takes place between administration of the first and the second dose or after administration of the second dose.

10. The method of, claim 1, wherein the immunogenic composition comprises one or more S. aureus antigen(s).

11. (canceled)

12. (canceled)

13. (canceled)

14. (canceled)

15. (canceled)

16. The method of claim 10, wherein the S. aureus antigen is selected from HlaH35L, EsxAB, Sta006 and Sta011.

17. (canceled)

18. (canceled)

19. (canceled)

20. (canceled)

21. (canceled)

22. The method of claim 1, wherein the immunogenic composition includes an adjuvant selected from an aluminium salt adjuvant, an oil-in-water emulsion adjuvant, and a TLR agonist.

23. The method of claim 1, wherein the subject is less than 1 year old.

24. The method of claim 1, wherein the immunogenic composition is administered by intramuscular injection.

25. (canceled)

26. The method of claim 1, wherein the immunomodulatory compound will be administered orally.

27. A method for enhancing the protective immunity elicited by an immunogen, wherein the method comprises administering an immunomodulatory compound to a subject who has previously been vaccinated with a composition comprising the immunogen, wherein one or more doses of the immunomodulatory compound is administered during a 24-48-hour time period before and/or after a second exposure to the immunogen.

28. The method of claim 27, wherein the second exposure is in the form of a live pathogen belonging to the same or a related species from which the immunogen was derived.

29. The method of claim 2, wherein the immunomodulatory compound is nicotinamide.


SEQUENCE LISTING (34 sequences)

SEQ ID NO: 1 - protein, 97 aa, Staphylococcus aureus
  SOURCE 1..97: /mol_type=“protein” /organism=“Staphylococcus aureus”
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 2 - protein, 104 aa, Staphylococcus aureus
  SOURCE 1..104: /mol_type=“protein” /organism=“Staphylococcus aureus”
MGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 3 - protein, 207 aa, artificial sequences
  SOURCE 1..207: /mol_type=“protein” /note=“EscAB fusion protein” /organism=“artificial sequences”
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSMGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 4 - protein, 207 aa, artificial sequences
  SOURCE 1..207: /mol_type=“protein” /note=“EsxBA fusion protein” /organism=“artificial sequences”
MGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNPASGGGSMAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 5 - protein, 6 aa, artificial sequences
  SOURCE 1..6: /mol_type=“protein” /note=“linker peptide” /organism=“artificial sequences”
ASGGGS

SEQ ID NO: 6 - protein, 205 aa, artificial sequences
  SOURCE 1..205: /mol_type=“protein” /note=“EsxAB fusion protein” /organism=“artificial sequences”
AMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 7 - protein, 206 aa, artificial sequences
  SOURCE 1..206: /mol_type=“protein” /note=“EsxAB fusion protein with N-terminal methionine” /organism=“artificial sequences”
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQASGGGSGGYKGIKADGGKVDQAKQLAAKTAKDIEACQKQTQQLAEYIEGSDWEGQFANKVKDVLLIMAKFQEELVQPMADHQKAIDNLSQNLAKYDTLSIKQGLDRVNP

SEQ ID NO: 8 - protein, 302 aa, Staphylococcus aureus
  SOURCE 1..302: /mol_type=“protein” /organism=“Staphylococcus aureus”
MKKLLLPLIIMLLVLAACGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 9 - protein, 285 aa, Artificial Sequence
  SOURCE 1..285: /mol_type=“protein” /note=“Sta006 mutant in which amino acids 1-17 have been deleted” /organism=“Artificial Sequence”
CGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 10 - protein, 288 aa, Artificial Sequence
  SOURCE 1..288: /mol_type=“protein” /note=“Sta006 mutant with N-terminal Met-Ala-Ser sequence” /organism=“Artificial Sequence”
MASCGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAK

SEQ ID NO: 11 - protein, 256 aa, Staphylococcus aureus
  SOURCE 1..256: /mol_type=“protein” /organism=“Staphylococcus aureus”
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 12 - protein, 233 aa, Artificial Sequence
  SOURCE 1..233: /mol_type=“protein” /note=“Sta011 mutant in which amino acids 1-23 have been deleted” /organism=“Artificial Sequence”
GCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 13 - protein, 234 aa, Artificial Sequence
  SOURCE 1..234: /mol_type=“protein” /note=“Sta011 deletion mutant with N-terminal methionine” /organism=“Artificial Sequence”
MGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKIKKEIENFKFFVQYGDFKNLKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 14 - protein, 256 aa, Artificial Sequence
  SOURCE 1..256: /mol_type=“protein” /note=“Sta011 substitution mutant” /organism=“Artificial Sequence”
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 15 - protein, 256 aa, Artificial Sequence
  SOURCE 1..256: /mol_type=“protein” /note=“Sta011 substitution mutant” /organism=“Artificial Sequence”
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKVKKEIENFKFFVQYGDFKNIKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 16 - protein, 256 aa, Artificial Sequence
  SOURCE 1..256: /mol_type=“protein” /note=“Sta011 substitution mutant” /organism=“Artificial Sequence”
MMKRLNKLVLGIIFLFLVISITAGCGIGKEAEVKKSFEKTLSMYPIKNLEDLYDKEGYRDDQFDKNDKGTWIINSEMVIQPNNEDMVAKGMVLYMNRNTKTTNGYYYVDVTKDEDEGKPHDNEKRYPVKMVDNKIIPTKEIKDEKLKKEIENFKFFVQYGDFKNVKNYKDGDISYNPEVPSYSAKYQLTNDDYNVKQLRKRYDIPTSKAPKLLLKGSGNLKGSSVGYKDIEFTFVEKKEENIYFSDSLDYKKSGDV

SEQ ID NO: 17 - protein, 319 aa, Staphylococcus aureus
  SOURCE 1..319: /mol_type=“protein” /organism=“Staphylococcus aureus”
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 18 - protein, 293 aa, Artificial Sequence
  SOURCE 1..293: /mol_type=“protein” /note=“Hla mutant in which amino acids 1-26 have been deleted” /organism=“Artificial Sequence”
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 19 - protein, 293 aa, Artificial Sequence
  SOURCE 1..293: /mol_type=“protein” /note=“Hla substitution mutant (H35L)” /organism=“Artificial Sequence”
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 20 - protein, 4 aa, Artificial Sequence
  SOURCE 1..4: /mol_type=“protein” /note=“loop replacement sequence” /organism=“Artificial Sequence”
PSGS

SEQ ID NO: 21 - protein, 258 aa, Artificial Sequence
  SOURCE 1..258: /mol_type=“protein” /note=“Hla loop replacement mutant (H35L)” /organism=“Artificial Sequence”
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTPSGSVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 22 - protein, 258 aa, Artificial Sequence
  SOURCE 1..258: /mol_type=“protein” /note=“Hla loop replacement mutant” /organism=“Artificial Sequence”
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTPSGSVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 23 - protein, 293 aa, Artificial Sequence
  SOURCE 1..293: /mol_type=“protein” /note=“Hla substitution mutant (Y101L)” /organism=“Artificial Sequence”
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDLYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 24 - protein, 293 aa, Artificial Sequence
  SOURCE 1..293: /mol_type=“protein” /note=“Hla substitution mutant (D152L)” /organism=“Artificial Sequence”
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPLFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 25 - protein, 293 aa, Artificial Sequence
  SOURCE 1..293: /mol_type=“protein” /note=“Hla substitution mutant (H35L, Y101L)” /organism=“Artificial Sequence”
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDLYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 26 - protein, 293 aa, Artificial Sequence
  SOURCE 1..293: /mol_type=“protein” /note=“Hla substitution mutant (H35L, D152L)” /organism=“Artificial Sequence”
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPLFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 27 - protein, 50 aa, Artificial Sequence
  SOURCE 1..50: /mol_type=“protein” /note=“Residues 27-76 of SEQ ID NO. 17” /organism=“Artificial Sequence”
ADSDINIKTGTTDIGSNTTVKTGDLVTYDKENGMHKKVFYSFIDDKNHNK

SEQ ID NO: 28 - protein, 63 aa, Artificial Sequence
  SOURCE 1..63: /mol_type=“protein” /note=“Residues 27-89 of SEQ ID NO. 17” /organism=“Artificial Sequence”
ADSDINIKTGTTDIGSNTTVKTGDLVTYDKENGMHKKVFYSFIDDKNHNKKLLVIRTKGTIAG

SEQ ID NO: 29 - protein, 53 aa, Artificial Sequence
  SOURCE 1..53: /mol_type=“protein” /note=“Residues 27-79 of SEQ ID NO. 17” /organism=“Artificial Sequence”
ADSDINIKTGTTDIGSNTTVKTGDLVTYDKENGMHKKVFYSFIDDKNHNKKLL

SEQ ID NO: 30 - protein, 50 aa, Artificial Sequence
  SOURCE 1..50: /mol_type=“protein” /note=“Residues 27-76 of SEQ ID NO. 19” /organism=“Artificial Sequence”
ADSDINIKTGTTDIGSNTTVKTGDLVTYDKENGMLKKVFYSFIDDKNHNK

SEQ ID NO: 31 - protein, 63 aa, Artificial Sequence
  SOURCE 1..63: /mol_type=“protein” /note=“Residues 27-89 of SEQ ID NO. 19” /organism=“Artificial Sequence”
ADSDINIKTGTTDIGSNTTVKTGDLVTYDKENGMLKKVFYSFIDDKNHNKKLLVIRTKGTIAG

SEQ ID NO: 32 - protein, 53 aa, Artificial Sequence
  SOURCE 1..53: /mol_type=“protein” /note=“Residues 27-79 of SEQ ID NO. 19” /organism=“Artificial Sequence”
ADSDINIKTGTTDIGSNTTVKTGDLVTYDKENGMLKKVFYSFIDDKNHNKKLL

SEQ ID NO: 33 - protein, 296 aa, Artificial Sequence
  SOURCE 1..296: /mol_type=“protein” /note=“Hla mutant” /organism=“Artificial Sequence”
MASADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 34 - DNA, 888 bp, Artificial Sequence
  source 1..888: /mol_type=“DNA” /note=“Expression vector for protein encoded by SEQ ID NO. 33” /organism=“Artificial Sequence”
atggctagcgcagattctgatattaatattaaaaccggtactacagatattggaagcaatactacagtaaaaacaggtgatttagtcacttatgataaagaaaatggcatgttaaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat